(19) United States
(12) Patent Application Publication
(10) Pub. No.: US 2026/0209203 A1
(43) Pub. Date: Jul. 23, 2026

(54) Heterocyclic Compounds as Modulators of S1P5

(71) Applicant: Celgene Corporation, Princeton, NJ (US)

(72) Inventors: Jean-Francois Brazeau, San Diego, CA (US); Rulin Ma, Winchester, MA (US); Jeffrey M. Schkeryantz, Summit, NJ (US); Karin Worm, Summit, NJ (US); Patrick W. Papa, Carlsbad, CA (US)

(73) Assignee: Celgene Corporation, Princeton, NJ (US)

(21) Appl. No.: 19/137,374

(22) PCT Filed: Dec. 14, 2023
(86) PCT No.: PCT/US2023/083982

§ 371 Date: Jun. 10, 2025

Related U.S. Application Data
(60) Provisional application No. 63/433,230, filed on Dec. 16, 2022.

(51) Int. Cl. C07D 401/10; A61K 31/4523; A61K 31/454; A61K 31/4545; A61K 31/496; A61K 31/5377; C07D 401/14

(52) U.S. Cl. CPC C07D 401/10; A61K 31/4523; A61K 31/454; A61K 31/4545; A61K 31/496; A61K 31/5377; C07D 401/14

(57) ABSTRACT

Provided herein are compounds and compositions thereof for modulating S1P5. In some embodiments, the compounds and compositions are provided for treatment of neurological diseases.

### CROSS-REFERENCE TO RELATED APPLICATIONS

This application claims priority to U.S. Provisional Application No. 63/433,230, filed on Dec. 16, 2022, which is incorporated herein by reference in its entirety for any purpose.

### FIELD

The present disclosure relates generally to compounds, compositions, and methods for their preparation and use of the compounds and compositions for treating neurological diseases.

### BACKGROUND

Sphingosine-1-phosphate (S1P; (2S,3R,4E)-2-amino-3-hydroxyoctadec-4-enyl-1-phosphate) is a bioactive sphingolipid that is synthesized by metabolic turnover of sphingolipids in cells and by the extracellular action of a secreted sphingosine kinase. S1P binds to and stimulates members of the endothelial cell differentiation gene family (EDG receptors), which are plasma membrane-localized G protein-coupled receptors. The five members of this family of receptors are S1P1 (EDG-1), S1P2 (EDG-5), S1P3 (EDG-3), S1P4 (EDG-6), and S1P5 (EDG-8). S1P mediates a wide variety of cellular responses including proliferation, cytoskeletal organization and migration, adherence- and tight junction assembly, and morphogenesis.

S1P5 is primarily expressed in the central nervous system. Specifically, S1P5 is highly expressed in oligodendrocytes (oligodendroglia) and oligodendrocyte progenitor cells (Jaillard, C. et al., J. Neuroscience, 2005, 25(6), 1459-1469; Novgorodov, A. S. et al., FASEB J., 2007, 21, 1503-1514). Oligodendrocytes are glial cells that form myelin sheaths (myelin) by binding to the axons of nerve cells. Compounds that bind to S1P5 can modulate the function of S1P5 and may be useful for treating neurodegenerative diseases.

Accordingly, in one aspect, provided herein are compounds that modulate S1P5 for use in treating neurodegenerative diseases.

### SUMMARY

Described herein, in certain embodiments, are compounds and compositions thereof for modulating S1P5. In various embodiments, the compounds and compositions thereof may be used for treatment of neurodegenerative diseases.

The present embodiments can be understood more fully by reference to the detailed description and examples, which are intended to exemplify non-limiting embodiments.

Embodiment A1 is a compound of Formula (I):

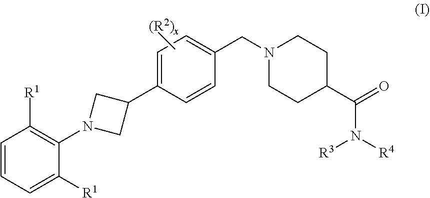

or a pharmaceutically acceptable salt thereof, wherein:
each R¹ is independently C₁-C₆ alkyl, C₃-C₆ cycloalkyl, or halo;
each R² is independently halo, C₁-C₆ alkyl, C₁-C₆ alkoxy, C₃-C₆ cycloalkyl, or C₁-C₆ haloalkyl;
x is 0-4;
R³ and R⁴ are each independently H, C₁-C₆ alkyl, C₁-C₆ alkyl-OH, —(C₁-C₆ alkylene)-O—(C₁-C₆ alkyl), C₃-C₆ cycloalkyl, —(C₁-C₆ alkylene)(C₃-C₆ cycloalkyl), or —(C₁-C₆ alkylene)NR^{5a}R^{5b} wherein each cycloalkyl is optionally substituted by 1-5 groups selected from halo, C₁-C₆ alkyl, C₁-C₆ haloalkyl, —OH, —NR^{6a}R^{6b}, and C₁-C₆ alkoxy;
or R³ and R⁴ are taken together with the nitrogen atom to which they are attached to form a 4- to 6-membered heterocyclyl optionally substituted with 1-5 R⁷ groups, wherein the heterocyclyl optionally contains 1-2 additional heteroatoms selected from N and O;
each R^{5a} and R^{5b} is independently H or C₁-C₆ alkyl;
or R^{5a} and R^{5b} are taken together with the nitrogen atom to which they are attached to form a 4- to 6-membered heterocyclyl;
each R^{6a} and R^{6b} is independently H or C₁-C₆ alkyl; and each R⁷ is independently halo, C₁-C₆ alkyl, —OH, C₁-C₆ alkoxy, or —NR^{6a}R^{6b}.

Embodiment A2 is the compound of embodiment A1, or a pharmaceutically acceptable salt thereof, wherein:
each R¹ is independently C₁-C₃ alkyl, C₃-C₆ cycloalkyl, or halo.

Embodiment A3 is the compound of embodiment A1 or A2, or a pharmaceutically acceptable salt thereof, wherein:
each R¹ is independently —CH₃, cyclopropyl, F, Cl, or Br.

Embodiment A4 is the compound of any one of embodiments A1-A3, or a pharmaceutically acceptable salt thereof, wherein:
each R¹ is Cl.

Embodiment A5 is the compound of any one of embodiments A1-A4, or a pharmaceutically acceptable salt thereof, wherein:
x is 1-4; and
each R² is independently halo, C₁-C₃ alkyl, C₁-C₃ alkoxy, C₃-C₆ cycloalkyl, or C₁-C₃ haloalkyl.

Embodiment A6 The compound of embodiment A5, or a pharmaceutically acceptable salt thereof, wherein:
x is 2 or 3; and
each R² is independently Cl, F, —CH₃, —OCH₃, cyclopropyl, or —CF₃.

Embodiment A7 is the compound of embodiment A6, or a pharmaceutically acceptable salt thereof, wherein:
x is 2; and
each R² is —CH₃.

Embodiment A8 is the compound of any one of embodiments A1-A7, or a pharmaceutically acceptable salt thereof, wherein:
R³ and R⁴ are independently H, C₁-C₅ alkyl, C₁-C₃ alkyl-OH, —(C₁-C₃ alkylene)-O—(C₁-C₃ alkyl), C₃-C₆ cycloalkyl, —(C₁-C₃ alkylene)(C₃-C₆ cycloalkyl), or —(C₁-C₃ alkylene)NR^{5a}R^{5b}, wherein each cycloalkyl is optionally substituted by 1-2 groups selected from halo, C₁-C₃ alkyl, C₁-C₃ haloalkyl, —OH, —NR^{6a}R^{6b}, and C₁-C₃ alkoxy;
each R^{5a} and R^{5b} is independently H or C₁-C₃ alkyl;
or R^{5a} and R^{5b} are taken together with the nitrogen atom to which they are attached to form a 5-to 6-membered heterocyclyl; and
each R^{6a} and R^{6b} is independently H or C₁-C₃ alkyl.

Embodiment A9 is the compound of embodiment A8, or a pharmaceutically acceptable salt thereof, wherein:
R³ and R⁴ are independently H, —CH₃, —CH(CH₃)₂, —CH₂CH(CH₃)₂, —CH₂CH₂CH(CH₃)₂, —CH₂CH₂OCH₃, —CH₂CH₂OH, —CH₂CH₂N(CH₃)₂, cyclopropyl, cyclobutyl, cyclopentyl, cyclohexyl, —CH₂(cyclopropyl), —CH₂(cyclohexyl), or —CH₂CH₂(pyrrolidinyl), wherein each cycloalkyl is optionally substituted with one group selected from —OH and —N(CH₃)₂.

Embodiment A10 is the compound of embodiment A9, or a pharmaceutically acceptable salt thereof, wherein:

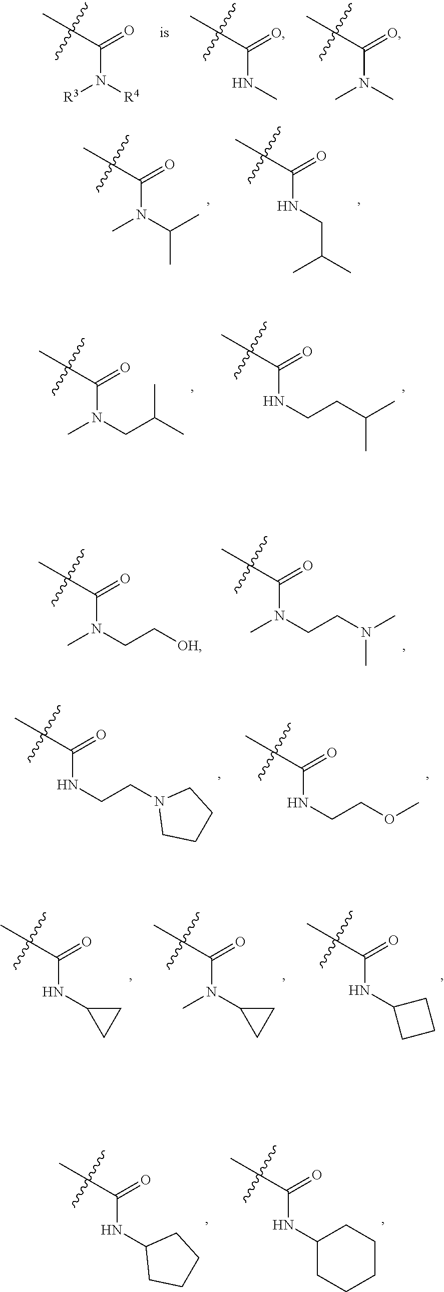

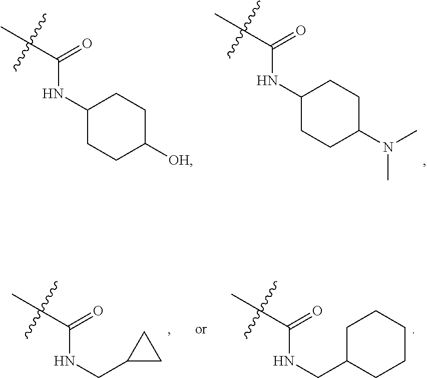 [SMILES: *C(=O)NC1CCC(O)CC1 ; *C(=O)NC1CCC(N(C)C)CC1 ; *C(=O)NCC1CC1 ; *C(=O)NCC1CCCCC1]

Embodiment A11 is the compound of any one of embodiments A1-A7, or a pharmaceutically acceptable salt thereof, wherein:
R³ and R⁴ are taken together with the nitrogen atom to which they are attached to form a 5- to 6-membered heterocyclyl optionally substituted with 1-2 R⁷ groups, wherein the heterocyclyl optionally contains one additional heteroatom selected from N and O;
each R⁷ is independently halo, C₁-C₃ alkyl, —OH, C₁-C₃ alkoxy, or —NR^{6a}R^{6b}; and each R^{6a} and R^{6b} is independently H or C₁-C₃ alkyl.

Embodiment A12 is the compound of embodiment A11, or a pharmaceutically acceptable salt thereof, wherein:
R³ and R⁴ are taken together with the nitrogen atom to which they are attached to form a 5- to 6-membered heterocyclyl optionally substituted with one R⁷ group, wherein the heterocyclyl optionally contains one additional heteroatom selected from N and O; and R⁷ is —OH, —CH₃, —OCH₃, or —N(CH₃)₂.

Embodiment A13 is the compound of embodiment A12, or a pharmaceutically acceptable salt thereof, wherein:

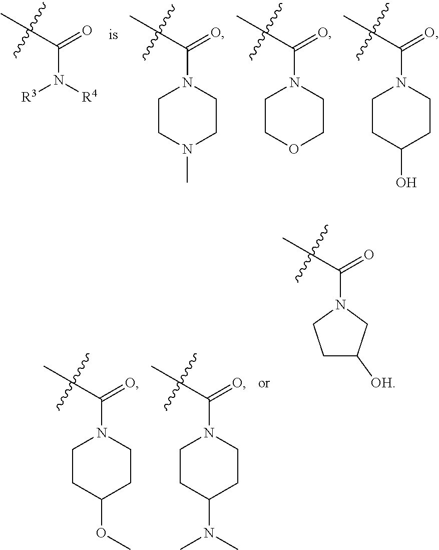

Embodiment A14 is the compound of any one of embodiments A1-A13, or a pharmaceutically acceptable salt thereof, wherein the compound is of Formula (11):

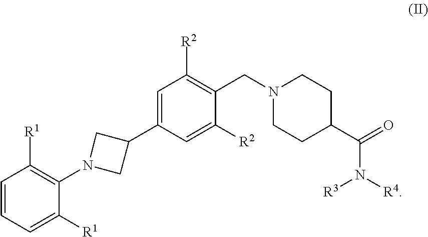

Embodiment A15 is the compound of any one of embodiments A1-A14, or a pharmaceutically acceptable salt thereof, wherein the compound is of Formula (IIIa):

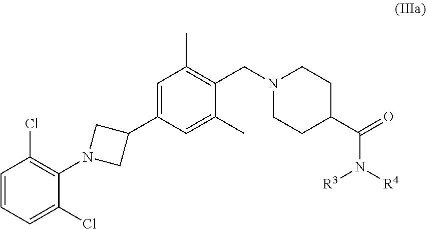

wherein:
R³ is H or C₁-C₆ alkyl;
R⁴ is H, C₁-C₆ alkyl, C₁-C₆ alkyl-OH, —(C₁-C₆ alkylene)-O—(C₁-C₆ alkyl), C₃-C₆ cycloalkyl, —(C₁-C₆ alkylene)(C₃-C₆ cycloalkyl), or —(C₁-C₆ alkylene)NR^{5a}R^{5b}, wherein each cycloalkyl is optionally substituted by 1-5 groups selected from halo, C₁-C₆ alkyl, C₁-C₆ haloalkyl, —OH, —NR^{6a}R^{6b}, and C₁-C₆ alkoxy; R^{5a} and R^{5b} are independently H or C₁-C₆ alkyl;
or R^{5a} and R^{5b} are taken together with the nitrogen atom to which they are attached to form a 4-to 6-membered heterocyclyl; and
each R^{6a} and R^{6b} is independently H or C₁-C₆ alkyl.

Embodiment A16 is the compound of any one of embodiments A1-A14, or a pharmaceutically acceptable salt thereof, wherein the compound is of Formula (IVa):

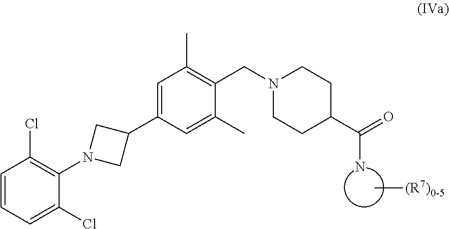

wherein

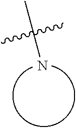 [SMILES: *N]

is a 4- to 6-membered heterocyclyl.

Embodiment A17 is a compound selected from the compounds of Table 1 and pharmaceutically acceptable salts thereof.

Embodiment A18 is a pharmaceutical composition comprising the compound of any one of embodiments A1-A17, or a pharmaceutically acceptable salt thereof, and a pharmaceutically acceptable excipient.

Embodiment A19 is a method of modulating sphingosine 1-phosphate receptor 5 (S1P5) comprising contacting S1P5 with an effective amount of the compound of any one of embodiments A1-A17, or a pharmaceutically acceptable salt thereof, or the pharmaceutical composition of embodiment A18.

Embodiment A20 is a method of treating a neurological disease in a subject in need thereof, comprising administering to the subject an effective amount of the compound of any one of embodiments A1-A17, or a pharmaceutically acceptable salt thereof, or the pharmaceutical composition of embodiment A18, optionally wherein the neurological disease is Alzheimer's disease or multiple sclerosis.

### DETAILED DESCRIPTION

### Definitions

As used herein, the terms “comprising” and “including” can be used interchangeably. The terms “comprising” and “including” are to be interpreted as specifying the presence of the stated features or components as referred to, but does not preclude the presence or addition of one or more features, or components, or groups thereof. Additionally, the terms “comprising” and “including” are intended to include examples encompassed by the term “consisting of”. Consequently, the term “consisting of” can be used in place of the terms “comprising” and “including” to provide for more specific embodiments of the invention.

The term “consisting of” means that a subject-matter has at least 90%, 95%, 97%, 98% or 99% of the stated features or components of which it consists. In another embodiment the term “consisting of” excludes from the scope of any succeeding recitation any other features or components, excepting those that are not essential to the technical effect to be achieved.

As used herein, the term “or” is to be interpreted as an inclusive “or” meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

In the present description, any concentration range, percentage range, ratio range, or integer range is to be understood to include the value of any integer within the recited range and, when appropriate, fractions thereof (such as one tenth and one hundredth of an integer), unless otherwise indicated. Also, any number range recited herein relating to any physical feature, such as polymer subunits, size, or thickness, are to be understood to include any integer within the recited range, unless otherwise indicated. As used herein, the terms “about” and “approximately” mean 20%, ±10%, ±5%, or ±1% of the indicated range, value, or structure, unless otherwise indicated.

An “alkyl” group is a saturated, partially saturated, or unsaturated straight chain or branched non-cyclic hydrocarbon having from 1 to 10 carbon atoms (C₁-C₁₀ alkyl), typically from 1 to 8 carbons (C₁-C₈ alkyl) or, in some embodiments, from 1 to 6 (C₁-C₆ alkyl), 1 to 3 (C₁-C₃ alkyl), or 2 to 6 (C₂-C₆ alkyl) carbon atoms. In some embodiments, the alkyl group is a saturated alkyl group. Representative saturated alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. In some embodiments, an alkyl group is an unsaturated alkyl group, also termed an alkenyl or alkynyl group. An “alkenyl” group is an alkyl group that contains one or more carbon-carbon double bonds. An “alkynyl” group is an alkyl group that contains one or more carbon-carbon triple bonds. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₂CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen; hydroxy; alkoxy; cycloalkyloxy, aryloxy, heterocyclyloxy, heteroaryloxy, heterocycloalkyloxy, cycloalkylalkyloxy, aralkyloxy, heterocyclylalkyloxy, heteroarylalkyloxy, heterocycloalkylalkyloxy; oxo (═O); amino, alkylamino, cycloalkylamino, arylamino, heterocyclylamino, heteroarylamino, heterocycloalkylamino, cycloalkylalkylamino, aralkylamino, heterocyclylalkylamino, heteroaralkylamino, heterocycloalkylalkylamino; imino; imido; amidino; guanidino; enamino; acylamino; sulfonylamino; urea, nitrourea; oxime; hydroxylamino; alkoxyamino; aralkoxyamino; hydrazino; hydrazido; hydrazono; azido; nitro; thio (—SH), alkylthio; ═S; sulfinyl; sulfonyl; aminosulfonyl; phosphonate; phosphinyl; acyl; formyl; carboxy; ester; carbamate; amido; cyano; isocyanato; isothiocyanato; cyanato; thiocyanato; or —B(OH)₂. In certain embodiments, when the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

An “alkyl-OH” group refers to an unbranched or branched alkyl group as defined above, wherein one or more hydrogen atoms are replaced by —OH. For example, “C₁-C₆ alkyl-OH” refers to a C₁-C₆ alkyl which is substituted by one or more —OH groups. An alkyl-OH may contain multiple hydroxy groups that are attached to the same carbon atom or to multiple carbon atoms.

An “alkylene” group refers to the same residues as alkyl, but having bivalency. Particular alkylene groups are those having from 1 to 10 carbon atoms (C₁-C₁₀ alkylene), typically from 1 to 8 carbons (C₁-C₈ alkylene) or, in some embodiments, from 1 to 6 (C₁-C₆ alkylene) or 1 to 3 (C₁-C₃ alkylene) carbon atoms. Examples of alkylene include, but are not limited to, groups such as methylene (—CH₂—), ethylene (—CH₂CH₂—), propylene (—CH₂CH₂CH₂—), isopropylene (—CH₂CH(CH₃)—), butylene (—CH₂(CH₂)₂CH₂—), isobutylene (—CH₂CH(CH₃)CH₂—), pentylene (—CH₂(CH₂)₃CH₂—), hexylene (—CH₂(CH₂)₄CH₂—), heptylene (—CH₂(CH₂)₅CH₂—), octylene (—CH₂(CH₂)₆CH₂—), and the like.

A “cycloalkyl” group is a saturated, or partially saturated cyclic alkyl group of from 3 to 10 carbon atoms (C₃-C₁₀ cycloalkyl) having a single cyclic ring or multiple condensed or bridged rings that can be optionally substituted. In some embodiments, the cycloalkyl group has 3 to 8 ring carbon atoms (C₃-C₈ cycloalkyl), whereas in other embodiments the number of ring carbon atoms ranges from 3 to 5 (C₃-C₅ cycloalkyl), 3 to 6 (C₃-C₆ cycloalkyl), or 3 to 7 (C₃-C₇ cycloalkyl). In some embodiments, the cycloalkyl groups are saturated cycloalkyl groups. Such saturated cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. In other embodiments, the cycloalkyl groups are unsaturated cycloalkyl groups. Examples of unsaturared cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

An “aryl” group is an aromatic carbocyclic group of from 6 to 14 carbon atoms (C₆-C₁₄ aryl) having a single ring (e.g., phenyl) or multiple condensed rings (e.g., naphthyl or anthryl). In some embodiments, aryl groups contain 6-14 carbons (C₆-C₁₄ aryl), and in others from 6 to 12 (C₆-C₁₂ aryl) or even 6 to 10 carbon atoms (C₆-C₁₀ aryl) in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “halogen” or “halo” is fluorine, chlorine, bromine or iodine.

“Haloalkyl” refers to an alkyl radical, as defined above, that is substituted by one or more halo radicals, as defined above, e.g., trifluoromethyl, difluoromethyl, trichloromethyl, 2,2,2-trifluoroethyl, 1,2-difluoroethyl, 3-bromo-2-fluoropropyl, 1,2-dibromoethyl, and the like. In some embodiments, the haloalkyl group has one to six carbon atoms and is substituted by one or more halo radicals (C₁-C₆ haloalkyl), or the haloalkyl group has one to three carbon atoms and is substituted by one or more halo radicals (C₁-C₃ haloalkyl). The halo radicals may be all the same or the halo radicals may be different. Unless specifically stated otherwise, a haloalkyl group is optionally substituted.

A “heteroaryl” group is an aromatic ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1H-benzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4-dihydroisoquinolin-1(2H)-onyl), tetrahydroquinolinyl, quinoxalinyl, and quinazolinyl groups. A heteroaryl group can be substituted or unsubstituted.

A “heterocyclyl” is a non-aromatic cycloalkyl in which one to four of the ring carbon atoms are independently replaced with a heteroatom selected from O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocycloalkyl group can be substituted or unsubstituted. Heterocyclyl groups encompass saturated and partially saturated ring systems. Further, the term heterocyclyl is intended to encompass any non-aromatic ring containing at least one heteroatom, which ring may be fused to an aryl or heteroaryl ring, regardless of the attachment to the remainder of the molecule. The phrase also includes bridged polycyclic ring systems containing a heteroatom. Representative examples of a heterocyclyl group include, but are not limited to, aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2(1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

An “alkoxy” group is —O-(alkyl), wherein alkyl is defined above.

A “carboxy” group is a radical of the formula: —C(O)OH.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonate; phosphine; thiocarbonyl; sulfinyl; sulfone; sulfonamide; ketone; aldehyde; ester; urea; urethane; oxime; hydroxyl amine; alkoxyamine; aralkoxyamine; N-oxide; hydrazine; hydrazide; hydrazone; azide; isocyanate; isothiocyanate; cyanate; thiocyanate; oxygen (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

Embodiments of the disclosure are meant to encompass pharmaceutically acceptable salts, tautomers, isotopologues, and stereoisomers of the compounds provided herein, such as the compounds of Formula (I).

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of formula (I) include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methyl-glucamine) and procaine. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, maleic, phosphoric, sulfuric, and methanesulfonic acids. Examples of specific salts thus include hydrochloride, formic, and mesylate salts. Others are well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereoisomerically pure” means one stereoisomer of a particular compound that is substantially free of other stereoisomers of that compound. For example, a stereoisomerically pure compound having one chiral center will be substantially free of the opposite enantiomer of the compound. A stereoisomerically pure compound having two chiral centers will be substantially free of other diastereomers of the compound. A typical stereoisomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds disclosed herein can have chiral centers and can occur as racemates, individual enantiomers or diastereomers, and mixtures thereof. All such isomeric forms are included within the embodiments disclosed herein, including mixtures thereof.

The use of stereoisomerically pure forms of the compounds disclosed herein, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); Wilen, S. H., Tables of Resolving Agents and Optical Resolutions p. 268 (E. L. Eliel, Ed., Univ. of Notre Dame Press, Notre Dame, IN, 1972); Todd, M., Separation Of Enantiomers: Synthetic Methods (Wiley-VCH Verlag GmbH & Co. KGaA, Weinheim, Germany, 2014); Toda, F., Enantiomer Separation: Fundamentals and Practical Methods (Springer Science & Business Media, 2007); Subramanian, G. Chiral Separation Techniques: A Practical Approach (John Wiley & Sons, 2008); Ahuj a, S., Chiral Separation Methods for Pharmaceutical and Biotechnological Products (John Wiley & Sons, 2011).

It should also be noted the compounds disclosed herein can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

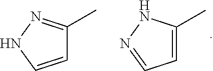

As readily understood by one skilled in the art, a wide variety of functional groups and other stuctures may exhibit tautomerism and all tautomers of compounds of Formula (I) are within the scope of the present disclosure.

It should also be noted the compounds disclosed herein can contain unnatural proportions of atomic isotopes at one or more of the atoms. For example, the compounds may be radiolabeled with radioactive isotopes, such as for example tritium (³H), iodine-125 (¹²⁵I) sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically encriched compounds are useful as therapeutic agents, e.g., cancer therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds disclosed herein, for example, the isotopologues are deuterium, carbon-13, and/or nitrogen-15 enriched compounds. As used herein, “deuterated”, means a compound wherein at least one hydrogen (H) has been replaced by deuterium (indicated by D or ²H), that is, the compound is enriched in deuterium in at least one position.

It is understood that, independently of stereoisomerical or isotopic composition, each compound disclosed herein can be provided in the form of any of the pharmaceutically acceptable salts discussed herein. Equally, it is understood that the isotopic composition may vary independently from the stereoisomerical composition of each compound referred to herein. Further, the isotopic composition, while being restricted to those elements present in the respective compound or salt thereof disclosed herein, may otherwise vary independently from the selection of the pharmaceutically acceptable salt of the respective compound.

It should be noted that if there is a discrepancy between a depicted structure and a name for that structure, the depicted structure is to be accorded more weight.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In one embodiment, the disorder is a neurodegenerative disease, as described herein, or a symptom thereof.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the disorder is a neurodegenerative disease, as described herein, or symptoms thereof.

The term “effective amount” in connection with a compound disclosed herein means an amount capable of treating or preventing a disorder, disease or condition, or symptoms thereof, disclosed herein.

The term “subject” or “patient” as used herein include an animal, including, but not limited to, an animal such a cow, monkey, horse, sheep, pig, chicken, turkey, quail, cat, dog, mouse, rat, rabbit or guinea pig, in one embodiment a mammal, in another embodiment a human. In one embodiment, a subject is a human having or at risk for having an S1P5 mediated disease, or a symptom thereof.

Although various features of the invention may be described in the context of a single embodiment, the features may also be provided separately or in any suitable combination. Conversely, although the invention may be described herein in the context of separate embodiments for clarity, the invention may also be implemented in a single embodiment.

### Compounds

In one aspect, provided herein is a compound of Formula (I):

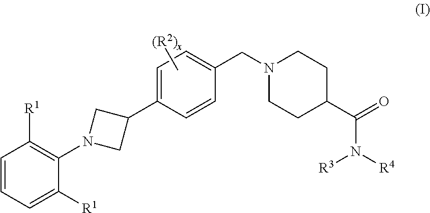

or a pharmaceutically acceptable salt thereof, wherein:
each R¹ is independently C₁-C₆ alkyl, C₃-C₆ cycloalkyl, or halo;
each R² is independently halo, C₁-C₆ alkyl, C₁-C₆ alkoxy, C₃-C₆ cycloalkyl, or C₁-C₆ haloalkyl;
x is 0-4;
R³ and R⁴ are each independently H, C₁-C₆ alkyl, C₁-C₆ alkyl-OH, —(C₁-C₆ alkylene)-O—(C₁-C₆ alkyl), C₃-C₆ cycloalkyl, —(C₁-C₆ alkylene)(C₃-C₆ cycloalkyl), or —(C₁-C₆ alkylene)NR^{5a}R^{5b}, wherein each cycloalkyl is optionally substituted by 1-5 groups selected from halo, C₁-C₆ alkyl, C₁-C₆ haloalkyl, —OH, —NR^{6a}R^{6b}, and C₁-C₆ alkoxy;
or R³ and R⁴ are taken together with the nitrogen atom to which they are attached to form a 4- to 6-membered heterocyclyl optionally substituted with 1-5 R⁷ groups, wherein the heterocyclyl optionally contains 1-2 additional heteroatoms selected from N and O;
each R^{5a} and R^{5b} is independently H or C₁-C₆ alkyl;
or R^{5a} and R^{5b} are taken together with the nitrogen atom to which they are attached to form a 4-to 6-membered heterocyclyl;
each R^{6a} and R^{6b} is independently H or C₁-C₆ alkyl; and each R⁷ is independently halo, C₁-C₆ alkyl, —OH, C₁-C₆ alkoxy, or —NR^{6a}R^{6b}.

In some embodiments, each R¹ is independently C₁-C₆ alkyl, C₃-C₆ cycloalkyl, or halo. In some embodiments, each R¹ is independently C₁-C₃ alkyl, C₃-C₆ cycloalkyl, F, Cl, Br, or I. In some embodiments, each R¹ is independently —CH₃, —CH₂CH₃, —CH₂CH₂CH₃, cyclopropyl, cyclobutyl, cyclopentyl, cyclohexyl, F, Cl, or Br. In some embodiments, each R¹ is independently methyl, cyclopropyl, F, Cl, or Br. In some embodiments, R¹ is Cl.

In some embodiments, R¹ is C₁-C₆ alkyl. In some embodiments, R¹ is C₁-C₃ alkyl. In some embodiments, R¹ is —CH₃, —CH₂CH₃, or —CH₂CH₂CH₃. In some embodiments, R¹ is —CH₃.

In some embodiments, R¹ is C₃-C₆ cycloalkyl. In some embodiments, R¹ is C₃-C₅ cycloalkyl. In some embodiments, R¹ is cyclopropyl, cyclobutyl, cyclopentyl, or cyclohexyl. In some embodiments, R¹ is cyclopropyl.

In some embodiments, R¹ is halo. In some embodiments, R¹ is F, Cl, Br, or I. In some embodiments, R¹ is F, Cl, or Br. In some embodiments, R¹ is Cl. In some embodiments, R¹ is F. In some embodiments, R¹ is Br.

In some embodiments, each R² is independently halo, C₁-C₆ alkyl, C₁-C₆ alkoxy, C₃-C₆ cycloalkyl, or C₁-C₆ haloalkyl. In some embodiments, each R² is independently halo, C₁-C₃ alkyl, C₁-C₃ alkoxy, C₃-C₆ cycloalkyl, or C₁-C₃ haloalkyl. In some embodiments, each R² is independently F, Cl, Br, I, —CH₃, —CH₂CH₃, —CH₂CH₂CH₃, —OCH₃, —OCH₂CH₃, —OCH₂CH₂CH₃, —OCH(CH₃)₂, cyclopropyl, cyclobutyl, cyclopentyl, cyclohexyl, or C₁ haloalkyl. In some embodiments, each R² is independently F, Cl, —CH₃, —OCH₃, cyclopropyl, —CH₂F, —CHF₂, or —CF₃. In some embodiments, each R² is independently F, Cl, —CH₃, —OCH₃, cyclopropyl, or —CF₃. In some embodiments, R² is —CH₃.

In some embodiments, R² is halo. In some embodiments, R² is F, Cl, Br, or I. In some embodiments, R² is F or Cl. In some embodiments, R² is F. In some embodiments, R² is Cl.

In some embodiments, R² is C₁-C₆ alkyl. In some embodiments, R² is C₁-C₃ alkyl. In some embodiments, R² is —CH₃, —CH₂CH₃, or —CH₂CH₂CH₃. In some embodiments, R² is —CH₃. In some embodiments, R² is —CH₂CH₃.

In some embodiments, R² is C₁-C₈alkoxy. In some embodiments, R² is C₁-C₃ alkoxy. In some embodiments, R² is —OCH₃, —OCH₂CH₃, —OCH₂CH₂CH₃, or —OCH(CH₃)₂. In some embodiments, R² is —OCH₃. In some embodiments, R² is —OCH₂CH₃.

In some embodiments, R² is C₃-C₆ cycloalkyl. In some embodiments, R² is cyclopropyl, cyclobutyl, cyclopentyl, or cyclohexyl. In some embodiments, R² is cyclopropyl.

In some embodiments, R² is C₁-C₆ haloalkyl. In some embodiments, R² is C₁-C₆ haloalkyl containing 1-13 halogen atoms. In some embodiments, R² is C₁-C₃ haloalkyl. In some embodiments, R² is C₁-C₃ haloalkyl containing 1-7 halogen atoms. In some embodiments, R² is —CF₃, —CHF₂, —CH₂F, —CCl₃, —CHCl₂, —CH₂Cl, —CF₂Cl, —CFCl₂, —CH₂CF₃, —CH₂CHF₂, or —CH₂CCl₃. In some embodiments, R² is C₁ haloalkyl. In some embodiments, R² is —CH₂F, —CHF₂, or —CF₃. In some embodiments, R² is —CF₃.

In some embodiments, x is 0-4. In some embodiments, x is 2 or 3. In some embodiments, x is 0. In some embodiments, x is 1. In some embodiments, x is 2. In some embodiments, x is 3. In some embodiments, x is 4.

In some embodiments, each R^{5a} and R^{5b} is independently H or C₁-C₆ alkyl. In some embodiments, each R^{5a} and R^{5b} is independently H or C₁-C₃ alkyl. In some embodiments, each R^{5a} and R^{5b} is independently H or —CH₃, —CH₂CH₃, or —CH₂CH₂CH₃. In some embodiments, each R^{5a} and R^{5b} is H or —CH₃. In some embodiments, each R^{5a} and R^{5b} is —CH₃.

In some embodiments, each R^{5a} and R^{5b} is H.

In some embodiments, each R^{5a} and R^{5b} is independently C₁-C₆ alkyl. In some embodiments, each R^{5a} and R^{5b} is independently C₁-C₃ alkyl. In some embodiments, each R^{5a} and R^{5b} is independently —CH₃, —CH₂CH₃, or —CH₂CH₂CH₃. In some embodiments, each R^{5a} and R^{5b} is —CH₃.

In some embodiments, one of R^{5a} and R^{5b} is H and the other of R^{5a} and R^{5b} is C₁-C₆ alkyl. In some embodiments, one of R^{5a} and R^{5b} is H and the other of R^{5a} and R^{5b} is C₁-C₃ alkyl, such as methyl, ethyl, or propyl. In some embodiments, one of R^{5a} and R^{5b} is H and the other of R^{5a} and R^{5b} is methyl.

In some embodiments, R^{5a} and R^{5b} are taken together with the nitrogen atom to which they are attached to form a 4- to 6-membered heterocyclyl. In some embodiments, R^{5a} and R^{5b} are taken together with the nitrogen atom to which they are attached to form a 5- to 6-membered heterocyclyl. In some embodiments, R^{5a} and R^{5b} are taken together with the nitrogen atom to which they are attached to form a 5-membered heterocyclyl. In some embodiments, R^{5a} and R^{5b} are taken together with the nitrogen atom to which they are attached to form an azetidinyl, pyrrolidinyl, or piperidinyl. In some embodiments, R^{5a} and R^{5b} are taken together with the nitrogen atom to which they are attached to form a pyrrolidinyl.

In some embodiments, each R^{6a} and R^{6b} is independently H or C₁-C₆ alkyl. In some embodiments, each R^{6a} and R^{6b} is independently H or C₁-C₃ alkyl. In some embodiments, each R^{6a} and R^{6b} is independently H, —CH₃, —CH₂CH₃, or —CH₂CH₂CH₃. In some embodiments, each R^{6a} and R^{6b} is independently H or —CH₃. In some embodiments, each R^{6a} and R^{6b} is —CH₃.

In some embodiments, each R^{6a} and R^{6b} is H.

In some embodiments, each R^{6a} and R^{6b} is independently C₁-C₆ alkyl. In some embodiments, each R^{6a} and R^{6b} is independently C₁-C₃ alkyl. In some embodiments, each R^{6a} and R^{6b} is independently —CH₃, —CH₂CH₃, or —CH₂CH₂CH₃. In some embodiments, each R^{6a} and R^{6b} is —CH₃.

In some embodiments, one of R^{6a} and R^{6b} is H and the other of R^{6a} and R^{6b} is C₁-C₆ alkyl. In some embodiments, one of R^{6a} and R^{6b} is H and the other of R^{6a} and R^{6b} is C₁-C₃ alkyl, such as methyl, ethyl, or propyl. In some embodiments, one of R^{6a} and R^{6b} is H and the other of R^{6a} and R^{6b} is methyl.

In some embodiments, each R⁷ is independently halo, C₁-C₆ alkyl, —OH, C₁-C₆ alkoxy, or —NR^{6a}R^{6b} In some embodiments, each R⁷ is halo, C₁-C₃ alkyl, —OH, C₁-C₃ alkoxy, or —NR^{6a}R^{6b} In some embodiments, each R⁷ is F, Cl, Br, I, —CH₃, —CH₂CH₃, —CH₂CH₂CH₃, —OH, —OCH₃, —OCH₂CH₃, —OCH₂CH₂CH₃, —OCH(CH₃)₂, or —NR^{6a}R^{6b}. In some embodiments, each R⁷ is —OH, —CH₃, —OCH₃, or —N(CH₃)₂. In some embodiments, each R⁷ is halo. In some embodiments, each R⁷ is F, Cl, Br, or I.

In some embodiments, R⁷ is C₁-C₆ alkyl. In some embodiments, R⁷ is C₁-C₃ alkyl. In some embodiments, R⁷ is —CH₃, —CH₂CH₃, or —CH₂CH₂CH₃. In some embodiments, R⁷ is —CH₃.

In some embodiments, R⁷ is —OH.

In some embodiments, R⁷ is C₁-C₃ alkoxy. In some embodiments, R⁷ is —OCH₃, —OCH₂CH₃, —OCH₂CH₂CH₃, or —OCH(CH₃)₂. In some embodiments, R⁷ is —OCH₃.

In some embodiments, R⁷ is —NR^{6a}R^{6b}. In some embodiments, R⁷ is —N(CH₃)₂. In some embodiments, R⁷ is —NH(CH₃).

In some embodiments, R³ and R⁴ are each independently H, C₁-C₆ alkyl, C₁-C₆ alkyl-OH, —(C₁-C₆ alkylene)-O—(C₁-C₆ alkyl), C₃-C₆ cycloalkyl, —(C₁-C₆ alkylene)(C₃-C₆ cycloalkyl), or —(C₁-C₆ alkylene)NR^{5a}R^{5b}, wherein each cycloalkyl is optionally substituted by 1-5 groups selected from halo, C₁-C₆ alkyl, C₁-C₆ haloalkyl, —OH, —NR^{6a}R^{6b}, and C₁-C₆ alkoxy. In some embodiments, R³ and R⁴ are each independently H, C₁-C₅ alkyl, C₁-C₃ alkyl-OH, —(C₁-C₃ alkylene)-O—(C₁-C₃ alkyl), C₃-C₆ cycloalkyl, —(C₁-C₃ alkylene)(C₃-C₆ cycloalkyl), or —(C₁-C₃ alkylene)NR^{5a}R^{5b}, wherein each cycloalkyl is optionally substituted by 1-5 groups selected from halo, C₁-C₆ alkyl, C₁-C₆ haloalkyl, —OH, —NR^{6a}R^{6b}, and C₁-C₆ alkoxy. In some embodiments, R³ and R⁴ are each independently H, C₁-C₅ alkyl, C₁-C₃ alkyl-OH, —(C₁-C₃ alkylene)-O—(C₁-C₃ alkyl), C₃-C₆ cycloalkyl, —(C₁-C₃ alkylene)(C₃-C₆ cycloalkyl), or —(C₁-C₃ alkylene)NR^{5a}R^{5b}, wherein each cycloalkyl is optionally substituted by 1-2 groups selected from halo, C₁-C₆ alkyl, C₁-C₆ haloalkyl, —OH, —NR^{6a}R^{6b}, and C₁-C₆ alkoxy. In some embodiments, R³ and R⁴ are each independently H, C₁-C₅ alkyl, C₁-C₃ alkyl-OH, —(C₁-C₃ alkylene)-O—(C₁-C₃ alkyl), C₃-C₆ cycloalkyl, —(C₁-C₃ alkylene)(C₃-C₆ cycloalkyl), or —(C₁-C₃ alkylene)NR^{5a}R^{5b}, wherein each cycloalkyl is optionally substituted by 1-2 groups selected from halo, C₁-C₃ alkyl, C₁-C₃ haloalkyl, —OH, —NR^{6a}R^{6b}, and C₁-C₃ alkoxy. In some embodiments, R³ and R⁴ are each independently H, C₁-C₅ alkyl, C₁-C₃ alkyl-OH, —(C₁-C₂ alkylene)-O—(C₁ alkyl), C₃-C₆ cycloalkyl, —(C₁ alkylene)(C₃-C₆ cycloalkyl), or —(C₂ alkylene)NR^{5a}R^{5b}, wherein each cycloalkyl is optionally substituted by 1-2 groups selected from halo, C₁-C₃ alkyl, C₁-C₃ haloalkyl, —OH, —NR^{6a}R^{6b}, and C₁-C₃ alkoxy. In some embodiments, R³ and R⁴ are each independently H, —CH₃, —CH₂CH₃, —CH₂CH₂CH₃, —CH(CH₃)₂, —CH₂CH₂CH₂CH₃, —C(CH₃)₃, —CH₂CH(CH₃)₂, —CH₂(CH₃)CH₂CH₃, —CH₂CH₂CH₂CH₂CH₃, —CH₂CH₂CH(CH₃)₂, —CH(CH₂CH₃)₂, —CH(CH₃)CH₂CH₂CH₃, —CH₂OH, —CH₂CH₂OH, —CH₂CH₂CH₂OH, —CH₂(CH₃)CH₂OH, —CH₂OCH₃, —CH₂CH₂OCH₃, cyclopropyl, cyclobutyl, cyclopentyl, cyclohexyl, —CH₂(cyclopropyl), —CH₂(cyclobutyl), —CH₂(cyclopentyl), —CH₂(cyclohexyl), —CH₂CH₂NR^{5a}R^{5b}, wherein each cycloalkyl is optionally substituted by 1-2 groups selected from halo, C₁-C₃ alkyl, C₁-C₃ haloalkyl, —OH, —NR^{6a}R^{6b} and C₁-C₃ alkoxy. In some embodiments, R³ and R⁴ are each independently H, —CH₃, —CH₂CH₃, —CH₂CH₂CH₃, —CH(CH₃)₂, —CH₂CH₂CH₂CH₃, —C(CH₃)₃, —CH₂CH(CH₃)₂, —CH₂(CH₃)CH₂CH₃, —CH₂CH₂CH₂CH₂CH₃, —CH₂CH₂CH(CH₃)₂, —CH(CH₂CH₃)₂, —CH(CH₃)CH₂CH₂CH₃, —CH₂OH, —CH₂CH₂OH, —CH₂CH₂CH₂OH, —CH₂(CH₃)CH₂OH, —CH₂OCH₃, —CH₂CH₂OCH₃, cyclopropyl, cyclobutyl, cyclopentyl, cyclohexyl, —CH₂(cyclopropyl), —CH₂(cyclobutyl), —CH₂(cyclopentyl), —CH₂(cyclohexyl), —CH₂CH₂NR^{5a}R^{5b}, wherein each cycloalkyl is optionally substituted by one group selected from halo, C₁-C₃ alkyl, C₁-C₃ haloalkyl, —OH, —NR^{6a}R^{6b}, and C₁-C₃ alkoxy. In some embodiments, R³ and R⁴ are each independently H, —CH₃, —CH(CH₃)₂, —CH₂CH(CH₃)₂, —CH₂CH₂CH(CH₃)₂, —CH₂CH₂OH, —CH₂CH₂OCH₃, cyclopropyl, cyclobutyl, cyclopentyl, cyclohexyl, —CH₂(cyclopropyl), —CH₂(cyclohexyl), —CH₂CH₂N(CH₃)₂, wherein each cycloalkyl is optionally substituted by one group selected from halo, C₁-C₃ alkyl, C₁ haloalkyl, —OH, —NR^{6a}R^{6b}, and C₁-C₃ alkoxy. In some embodiments, R³ and R⁴ are each independently H, —CH₃, —CH(CH₃)₂, —CH₂CH(CH₃)₂, —CH₂CH₂CH(CH₃)₂, —CH₂CH₂OH, —CH₂CH₂OCH₃, cyclopropyl, cyclobutyl, cyclopentyl, cyclohexyl, —CH₂(cyclopropyl), —CH₂(cyclohexyl), —CH₂CH₂N(CH₃)₂, wherein each cycloalkyl is optionally substituted by one group selected from F, Cl, Br, I, —CH₃, —CH₂CH₃, —CH₂CH₂CH₃, —CH(CH₃)₂, —CH₂F, —CHF₂, —CF₃, —OH, —NR^{6a}R^{6b}, —OCH₃, —OCH₂CH₃, —OCH₂CH₂CH₃, and —OCH(CH₃)₂. In some embodiments, R³ and R⁴ are each independently H, —CH₃, —CH(CH₃)₂, —CH₂CH(CH₃)₂, —CH₂CH₂CH(CH₃)₂, —CH₂CH₂OH, —CH₂CH₂OCH₃, cyclopropyl, cyclobutyl, cyclopentyl, cyclohexyl, —CH₂(cyclopropyl), —CH₂(cyclohexyl), —CH₂CH₂N(CH₃)₂, wherein each cycloalkyl is optionally substituted by one group selected from —OH and —N(CH₃)₂.

In some embodiments,

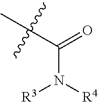

is:

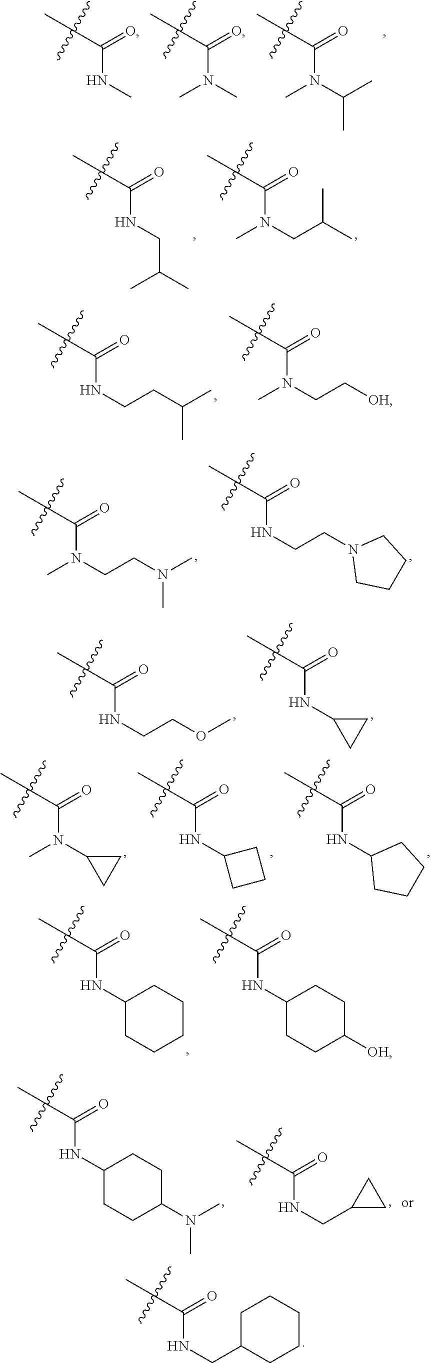

In some embodiments, R³ and R⁴ are taken together with the nitrogen atom to which they are attached to form a 4- to 6-membered heterocyclyl optionally substituted with 1-5 R⁷ groups, wherein the heterocyclyl optionally contains 1-2 additional heteroatoms selected from N and O. In some embodiments, R³ and R⁴ are taken together with the nitrogen atom to which they are attached to form a 5- to 6-membered heterocyclyl optionally substituted with 1-5 R⁷ groups, wherein the heterocyclyl optionally contains 1-2 additional heteroatoms selected from N and O. In some embodiments, R³ and R⁴ are taken together with the nitrogen atom to which they are attached to form a 5- to 6-membered heterocyclyl optionally substituted with 1-2 R⁷ groups, wherein the heterocyclyl optionally contains one additional heteroatom selected from N and O. In some embodiments, R³ and R⁴ are taken together with the nitrogen atom to which they are attached to form a 5- to 6-membered heterocyclyl optionally substituted with one R⁷ group, wherein the heterocyclyl optionally contains one additional heteroatom selected from N and O. In some embodiments, R³ and R⁴ are taken together with the nitrogen atom to which they are attached to form a 5- to 6-membered heterocyclyl optionally substituted with one R⁷ group, wherein the heterocyclyl optionally contains one additional heteroatom selected from N and O; wherein R⁷ is —OH, —CH₃, —OCH₃, or —N(CH₃)₂. In some embodiments, the heterocyclyl is azetidinyl, pyrrolidinyl, piperidinyl, piperazinyl, or morpholinyl.

In some embodiments,

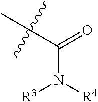

is:

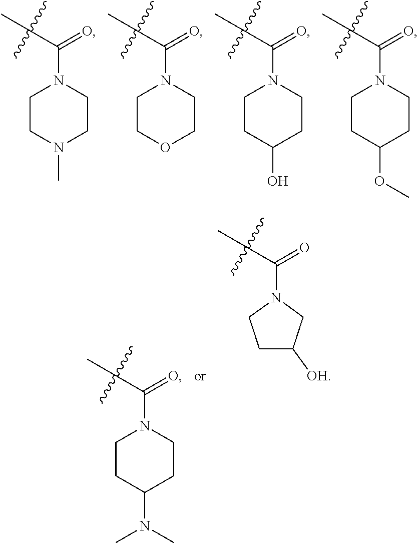

In some embodiments, the compound of Formula (I) is a compound of Formula (II):

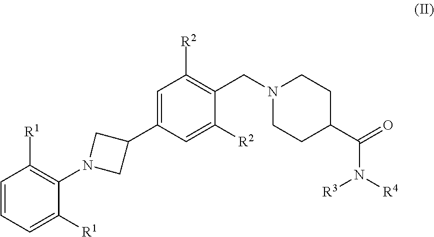

wherein R¹, R², R³, and R⁴ are as described for Formula (I).

In some embodiments, the compound of Formula (I) is a compound of Formula (III):

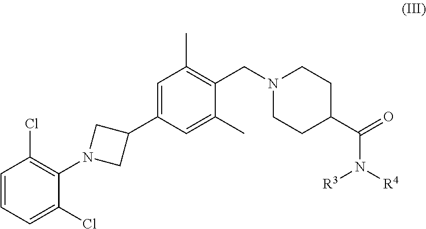

wherein R³ and R⁴ are as described for Formula (I).

In some embodiments, the compound of Formula (I) is a compound of Formula (IIIa):

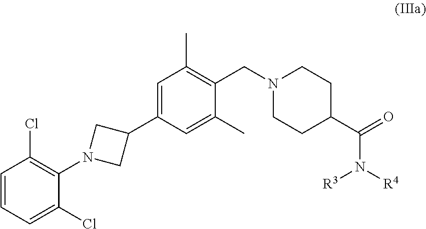

wherein:
R³ is H or C₁-C₆ alkyl;
R⁴ is H, C₁-C₆ alkyl, C₁-C₆ alkyl-OH, —(C₁-C₆ alkylene)-O—(C₁-C₆ alkyl), C₃-C₆ cycloalkyl, —(C₁-C₆ alkylene)(C₃-C₆ cycloalkyl), or —(C₁-C₆ alkylene)NR^{5a}R^{5b}, wherein each cycloalkyl is optionally substituted by 1-5 groups selected from halo, C₁-C₆ alkyl, C₁-C₆ haloalkyl, —OH, —NR^{6a}R^{6b}, and C₁-C₆ alkoxy;
R^{5a} and R^{5b} are independently H or C₁-C₆ alkyl;
or R^{5a} and R^{5b} are taken together with the nitrogen atom to which they are attached to form a 4-to 6-membered heterocyclyl; and
each R^{6a} and R^{6b} is independently H or C₁-C₆ alkyl.

In some embodiments, the compound of Formula (I) is a compound of Formula (IIIb) or Formula (IIIc):

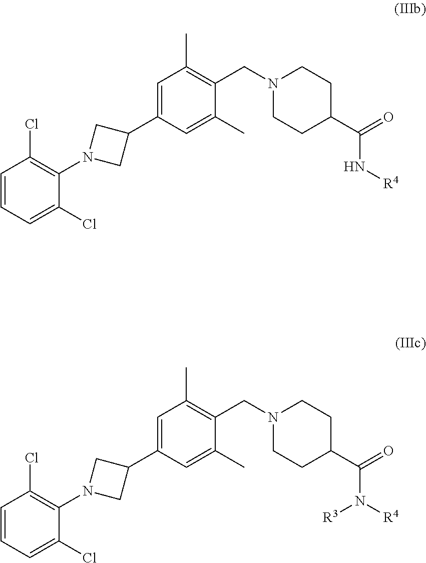

wherein:
R³ is C₁-C₆ alkyl;
R⁴ is H, C₁-C₆ alkyl, C₁-C₆ alkyl-OH, —(C₁-C₆ alkylene)-O—(C₁-C₆ alkyl), C₃-C₆ cycloalkyl, —(C₁-C₆ alkylene)(C₃-C₆ cycloalkyl), or —(C₁-C₆ alkylene)NR^{5a}R^{5b}, wherein each cycloalkyl is optionally substituted by 1-5 groups selected from halo, C₁-C₆ alkyl, C₁-C₆ haloalkyl, —OH, —NR^{6a}R^{6b}, and C₁-C₆ alkoxy;
R^{5a} and R^{5b} are independently H or C₁-C₆ alkyl;
or R^{5a} and R^{5b} are taken together with the nitrogen atom to which they are attached to form a 4-to 6-membered heterocyclyl; and
each R^{6a} and R^{6b} is independently H or C₁-C₆ alkyl

In some embodiments, the compound of Formula (I) is a compound of Formula (IV):

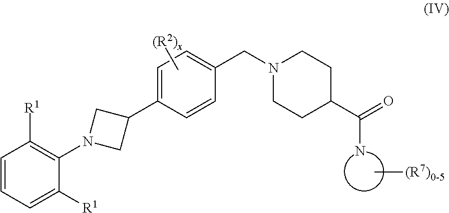

wherein R¹, R², R⁷, and x are as described for Formula (I); and

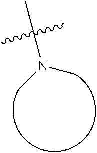

is a 4- to 6-membered heterocyclyl.

In some embodiments, the compound of Formula (I) is a compound of Formula (IVa):

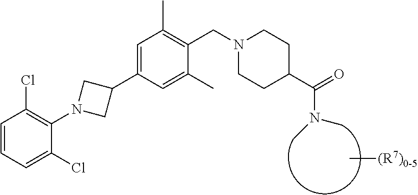

wherein R⁷ is as described for Formula (I);

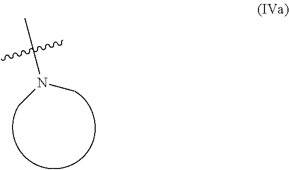

is a 4- to 6-membered heterocyclyl.

In the descriptions herein, it is understood that every description, variation, embodiment, or aspect of a moiety may be combined with every description, variation, embodiment, or aspect of other moieties the same as if each and every combination of descriptions is specifically and individually listed. For example, every description, variation, embodiment, or aspect provided herein with respect to R¹ of Formula (I) may be combined with every description, variation, embodiment, or aspect of R², R³, R⁴, R^{5a}, R^{5b}, R^{6a}, R^{6b}, R⁷, and x, the same as if each and every combination were specifically and individually listed. It is also understood that all descriptions, variations, embodiments, or aspects of Formula (I), where applicable, apply equally to other formulae detailed herein, and are equally described, the same as if each and every description, variation, embodiment, or aspect were separately and individually listed for all formulae. For example, all descriptions, variations, embodiments, or aspects of Formula (I), where applicable, apply equally to any of the formulae as detailed herein, such as Formulae (II), (III), (IIIa), (IIIb), (IIIc) (IV), and (IVa), and are equally described, the same as if each and every description, variation, embodiment, or aspect were separately and individually listed for all formulae.

In some embodiments, provided is a compound selected from the compounds in Table 1 or a pharmaceutically acceptable salt thereof. It is understood that any or all stereochemical forms, including any enantiomeric or diastereomeric forms, and any tautomers or other forms of any of the compounds of the present disclosure, including in Table 1, are herein described.

TABLE 1

| Compound | | |
|---|---|---|
| No. | Structure | Name |
| 1 | 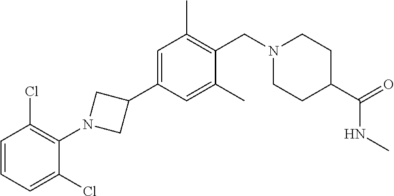 [SMILES: CNC(=O)C1CCN(Cc2c(C)cc(C3CN(c4c(Cl)cccc4Cl)C3)cc2C)CC1] | 1-(4-(1-(2,6-dichlorophenyl)- azetidin-3-yl)-2,6-dimethyl- benzyl)-N-methylpiperidine- 4-carboxamide |
| 2 | 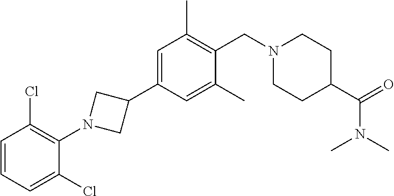 [SMILES: Cc1cc(C2CN(c3c(Cl)cccc3Cl)C2)cc(C)c1CN1CCC(C(=O)N(C)C)CC1] | 1-(4-(1-(2,6-dichlorophenyl)- azetidin-3-yl)-2,6-dimethyl- benzyl)-N,N-dimethylpiper- idine-4-carboxamide |
| 3 | 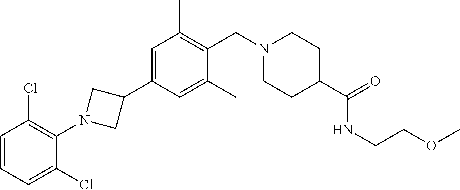 [SMILES: COCCNC(=O)C1CCN(Cc2c(C)cc(C3CN(c4c(Cl)cccc4Cl)C3)cc2C)CC1] | 1-(4-(1-(2,6-dichlorophenyl)- azetidin-3-yl)-2,6-dimethyl- benzyl)-N-(2-methoxyethyl)- piperidine-4-carboxamide |
| 4 | 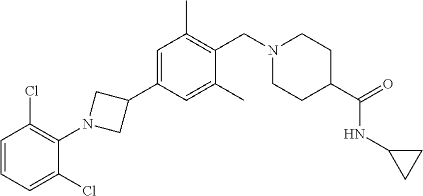 [SMILES: Cc1cc(C2CN(c3c(Cl)cccc3Cl)C2)cc(C)c1CN1CCC(C(=O)NC2CC2)CC1] | N-cyclopropyl-1-(4-(1-(2,6- dichlorophenyl)-azetidin-3- yl)-2,6-dimethylbenzyl)- piperidine-4-carboxamide |
| 5 | 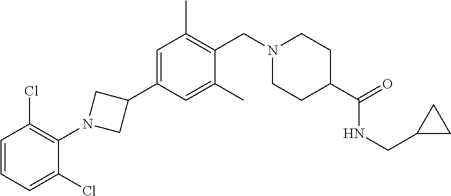 [SMILES: Cc1cc(C2CN(c3c(Cl)cccc3Cl)C2)cc(C)c1CN1CCC(C(=O)NCC2CC2)CC1] | N-(cyclopropylmethyl)-1-(4- (1-(2,6-dichlorophenyl)- azetidin-3-yl)-2,6-dimethyl- benzyl)piperidine-4-carbox- amide |
| 6 | 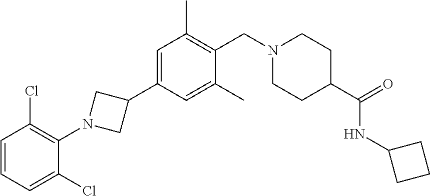 [SMILES: Cc1cc(C2CN(c3c(Cl)cccc3Cl)C2)cc(C)c1CN1CCC(C(=O)NC2CCC2)CC1] | N-cyclobutyl-1-(4-(1-(2,6- dichlorophenyl)azetidin-3- yl)-2,6-dimethylbenzyl)- piperidine-4-carboxamide |
| 7 | 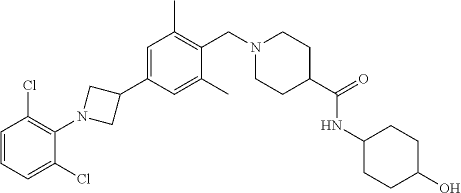 [SMILES: Cc1cc(C2CN(c3c(Cl)cccc3Cl)C2)cc(C)c1CN1CCC(C(=O)NC2CCC(O)CC2)CC1] | 1-(4-(1-(2,6-dichlorophenyl)- azetidin-3-yl)-2,6-dimethyl- benzyl)-N-(4-hydroxycyclo- hexyl)-piperidine-4-carbox- amide |
| 8 | 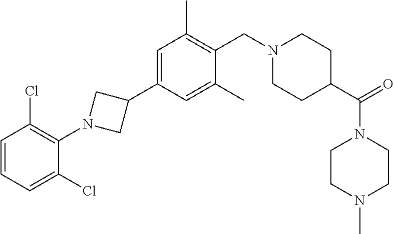 [SMILES: Cc1cc(C2CN(c3c(Cl)cccc3Cl)C2)cc(C)c1CN1CCC(C(=O)N2CCN(C)CC2)CC1] | (1-(4-(1-(2,6-dichlorophen- yl)azetidin-3-yl)-2,6-dimeth- ylbenzyl)-piperidin-4-yl)(4- methyl-piperazin-1-yl)- methanone |
| 9 | 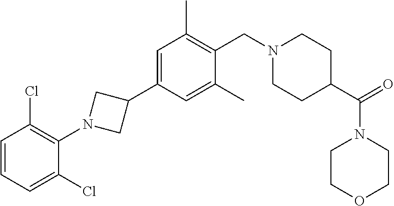 [SMILES: Cc1cc(C2CN(c3c(Cl)cccc3Cl)C2)cc(C)c1CN1CCC(C(=O)N2CCOCC2)CC1] | (1-(4-(1-(2,6-dichlorophen- yl)azetidin-3-yl)-2,6-dimeth- ylbenzyl)-piperidin-4-yl)- (morpholino)methanone |
| 10 | 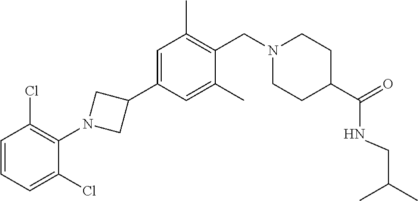 [SMILES: Cc1cc(C2CN(c3c(Cl)cccc3Cl)C2)cc(C)c1CN1CCC(C(=O)NCC(C)C)CC1] | 1-(4-(1-(2,6-dichlorophenyl)- azetidin-3-yl)-2,6-dimethyl- benzyl)-N-isobutylpiperidine- 4-carboxamide |
| 11 | 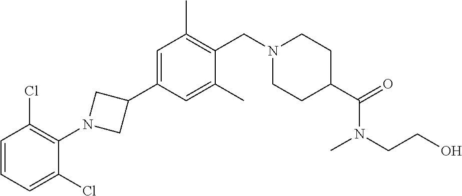 [SMILES: Cc1cc(C2CN(c3c(Cl)cccc3Cl)C2)cc(C)c1CN1CCC(C(=O)N(C)CCO)CC1] | 1-(4-(1-(2,6-dichlorophenyl)- azetidin-3-yl)-2,6-dimethyl- benzyl)-N-(2-hydroxyethyl)- N-methylpiperidine-4-carbox- amide |
| 12 | 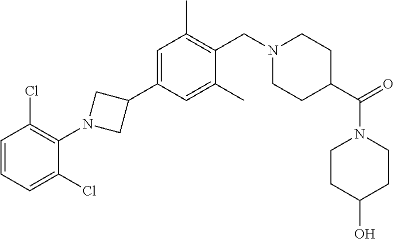 [SMILES: Cc1cc(C2CN(c3c(Cl)cccc3Cl)C2)cc(C)c1CN1CCC(C(=O)N2CCC(O)CC2)CC1] | (1-(4-(1-(2,6-dichlorophenyl)- azetidin-3-yl)-2,6-dimethyl- benzyl)-piperidin-4-yl)(4- hydroxy-piperidin-1-yl)- methanone |
| 13 | 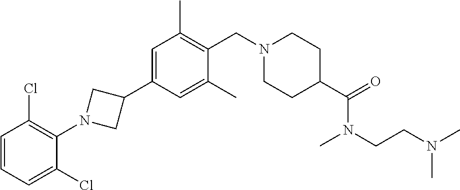 [SMILES: Cc1cc(C2CN(c3c(Cl)cccc3Cl)C2)cc(C)c1CN1CCC(C(=O)N(C)CCN(C)C)CC1] | 1-(4-(1-(2,6-dichlorophenyl)- azetidin-3-yl)-2,6-dimethyl- benzyl)-N-(2-(dimethyl- amino)ethyl)-N-methylpiper- idine-4-carboxamide |
| 14 | 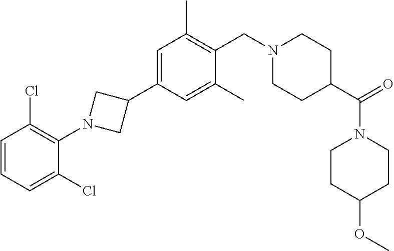 [SMILES: COC1CCN(C(=O)C2CCN(Cc3c(C)cc(C4CN(c5c(Cl)cccc5Cl)C4)cc3C)CC2)CC1] | (1-(4-(1-(2,6-dichlorophenyl)- azetidin-3-yl)-2,6-dimethyl- benzyl)-piperidin-4-yl)(4- methoxy-piperidin-1-yl)- methanone |
| 15 | 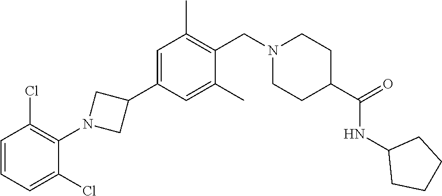 [SMILES: Cc1cc(C2CN(c3c(Cl)cccc3Cl)C2)cc(C)c1CN1CCC(C(=O)NC2CCCC2)CC1] | N-cyclopentyl-1-(4-(1-(2,6- dichlorophenyl)-azetidin-3- yl)-2,6-dimethylbenzyl)- piperidine-4-carboxamide |
| 16 | 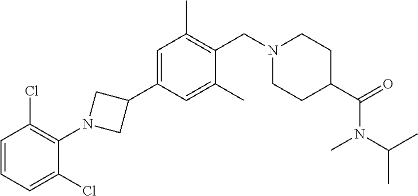 [SMILES: Cc1cc(C2CN(c3c(Cl)cccc3Cl)C2)cc(C)c1CN1CCC(C(=O)N(C)C(C)C)CC1] | 1-(4-(1-(2,6-dichlorophenyl)- azetidin-3-yl)-2,6-dimethyl- benzyl)-N-isopropyl-N-meth- yl-piperidine-4-carboxamide |
| 17 | 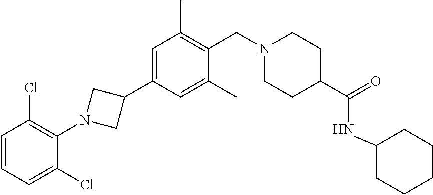 [SMILES: Cc1cc(C2CN(c3c(Cl)cccc3Cl)C2)cc(C)c1CN1CCC(C(=O)NC2CCCCC2)CC1] | N-cyclohexyl-1-(4-(1-(2,6- dichlorophenyl)azetidin-3- yl)-2,6-dimethylbenzyl)- piperidine-4-carboxamide |
| 18 | 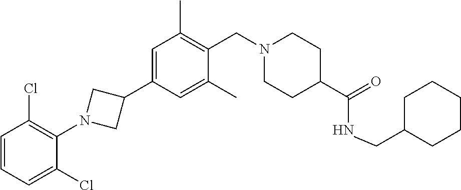 [SMILES: Cc1cc(C2CN(c3c(Cl)cccc3Cl)C2)cc(C)c1CN1CCC(C(=O)NCC2CCCCC2)CC1] | N-(cyclohexylmethyl)-1-(4- (1-(2,6-dichlorophenyl)- azetidin-3-yl)-2,6-dimethyl- benzyl)piperidine-4-carbox- amide |
| 19 | 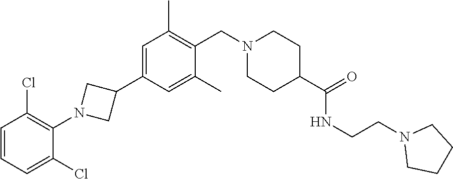 [SMILES: Cc1cc(C2CN(c3c(Cl)cccc3Cl)C2)cc(C)c1CN1CCC(C(=O)NCCN2CCCC2)CC1] | 1-(4-(1-(2,6-dichlorophenyl)- azetidin-3-yl)-2,6-dimethyl- benzyl)-N-(2-(pyrrolidin-1- yl)ethyl)-piperidine-4-carbox- amide |
| 20 | 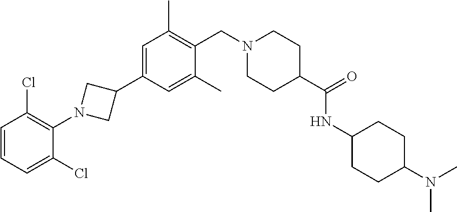 [SMILES: Cc1cc(C2CN(c3c(Cl)cccc3Cl)C2)cc(C)c1CN1CCC(C(=O)NC2CCC(N(C)C)CC2)CC1] | 1-(4-(1-(2,6-dichlorophenyl)- azetidin-3-yl)-2,6-dimethyl- benzyl)-N-(4-(dimethyl- amino)-cyclohexyl)piper- idine-4-carboxamide |
| 21 | 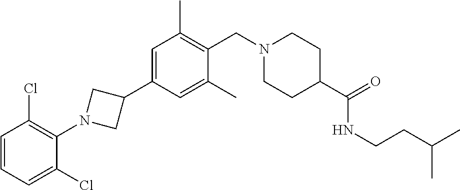 [SMILES: Cc1cc(C2CN(c3c(Cl)cccc3Cl)C2)cc(C)c1CN1CCC(C(=O)NCCC(C)C)CC1] | 1-(4-(1-(2,6-dichlorophenyl)- azetidin-3-yl)-2,6-dimethyl- benzyl)-N-isopentyl-piper- idine-4-carboxamide |
| 22 | 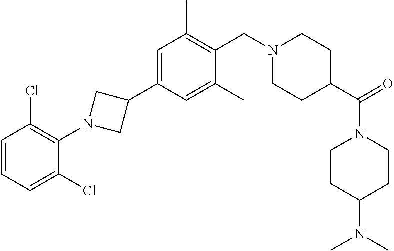 [SMILES: Cc1cc(C2CN(c3c(Cl)cccc3Cl)C2)cc(C)c1CN1CCC(C(=O)N2CCC(N(C)C)CC2)CC1] | (1-(4-(1-(2,6-dichlorophenyl)- azetidin-3-yl)-2,6-dimethyl- benzyl)piperidin-4-yl)(4- (dimethylamino)piperidin-1- yl)methanone |
| 23 | 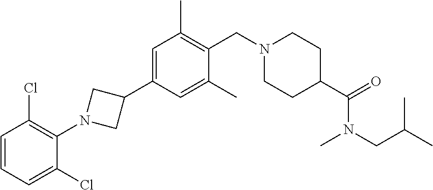 [SMILES: Cc1cc(C2CN(c3c(Cl)cccc3Cl)C2)cc(C)c1CN1CCC(C(=O)N(C)CC(C)C)CC1] | 1-(4-(1-(2,6-dichlorophenyl)- azetidin-3-yl)-2,6-dimethyl- benzyl)-N-isobutyl-N-meth- ylpiperidine-4-carboxamide |
| 24 | 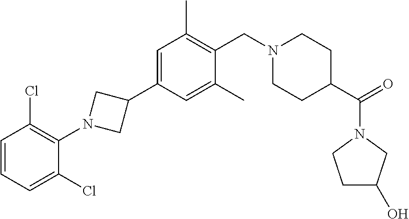 [SMILES: Cc1cc(C2CN(c3c(Cl)cccc3Cl)C2)cc(C)c1CN1CCC(C(=O)N2CCC(O)C2)CC1] | (1-(4-(1-(2,6-dichlorophenyl)- azetidin-3-yl)-2,6-dimethyl- benzyl)-piperidin-4-yl)(3- hydroxypyrrolidin-1-yl)- methanone |
| 25 | 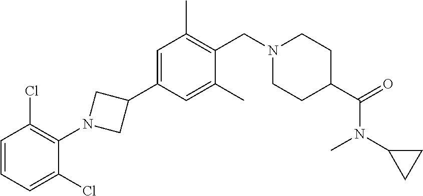 [SMILES: Cc1cc(C2CN(c3c(Cl)cccc3Cl)C2)cc(C)c1CN1CCC(C(=O)N(C)C2CC2)CC1] | N-cyclopropyl-1-(4-(1-(2,6- dichlorophenyl)-azetidin-3- yl)-2,6-dimethylbenzyl)-N- methylpiperidine-4-carbox- amide |
| 26 | 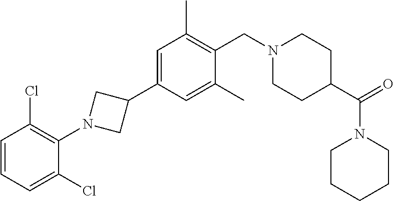 [SMILES: Cc1cc(C2CN(c3c(Cl)cccc3Cl)C2)cc(C)c1CN1CCC(C(=O)N2CCCCC2)CC1] | (1-(4-(1-(2,6-dichlorophenyl)- azetidin-3-yl)-2,6-dimethyl- benzyl)-piperidin-4-yl)(piper- idin-1-yl)methanone |

or a pharmaceutically acceptable salt thereof.

It is understood that in the present description, combinations of substituents and/or variables of the depicted formulae are permissible only if such contributions result in stable compounds.

Furthermore, all compounds of Formula (I) that exist in free base or acid form can be converted to their pharmaceutically acceptable salts by treatment with the appropriate inorganic or organic base or acid by methods known to one skilled in the art. Salts of the compounds of Formula (I) can be converted to their free base or acid form by standard techniques.

### Methods of Synthesis

The compounds described herein can be made using conventional organic syntheses and commercially available starting materials, or the methods provided herein. By way of example and not limitation, compounds of Formula (I) can be prepared as outlined in Scheme 1 and Scheme 2, as well as in the examples set forth herein. It should be noted that one skilled in the art would know how to modify the procedures set forth in the illustrative schemes and examples to arrive at the desired products.

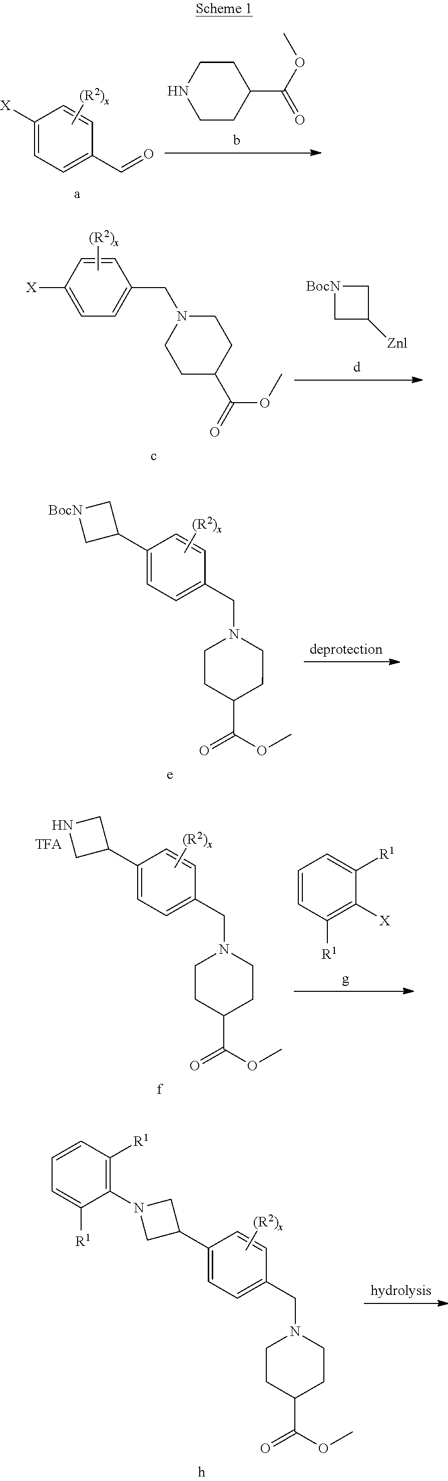

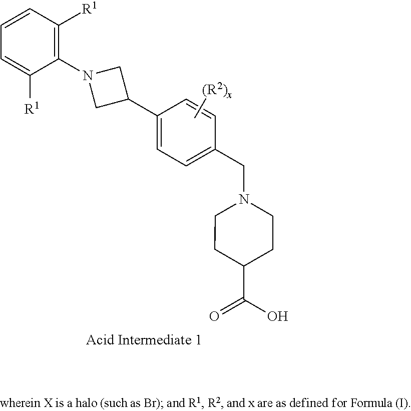

Scheme 1 shows a synthetic route to Acid Intermediate I, which can be used to prepare compounds of Formula (I). Reductive amination of a and b affords c, which is then coupled with d under, for example, Palladium catalyzed conditions, to generate e. Acid deprotection of e affords amine f, which is coupled with g to yield h. In a final step, ester h is hydrolyzed to give Acid Intermediate 1.

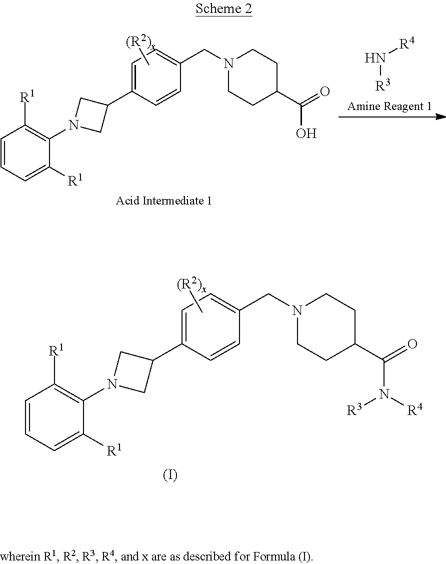

Scheme 2 shows a synthetic route to compounds of Formula (I). Acid Intermediate 1 is coupled with Amine Reagent 1 using, for example, HATU, to give compounds of Formula (I).

### Methods of Use

Embodiments of the present disclosure provide a method for modulating sphingosine 1-phosphate receptor 5 (S1P5) in a subject in need thereof, the method comprising administering to the subject an effective amount of a compound of Formula (I). Modulation (e.g., inhibition or activation) of S1P5 can be assessed and demonstrated by a wide variety of ways known in the art. Kits and commercially available assays can be utilized for determining whether and to what degree S1P5 has been modulated (e.g., inhibited or activated).

In one aspect, provided herein is a method of modulating S1P5 comprising contacting S1P5 with an effective amount of a compound of Formula (I) or any embodiment or variation thereof. In some embodiments, the compound of Formula (I) inhibits S1P5. In other embodiments, the compound of Formula (I) activates S1P5. In some embodiments, the compound of Formula (I) is an agonist of S1P5. In some embodiments, the compound of Formula (I) is an antagonist of S1P5.

In some embodiments, a compound of Formula (I) modulates the activity of S1P5 by about 1%, 5%, 10%, 15%, 20%, 25%, 30%, 35%, 40%, 45%, 50%, 55%, 60%, 65%, 70%, 75%, 80%, 85%, 90%, 95%, or 100%. In some embodiments, a compound of Formula (I) modulates the activity of S1P5 by about 1-100%, 5-100%, 10-100%, 15-100%, 20-100%, 25-100%, 30-100%, 35-100%, 40-100%, 45-100%, 50-100%, 55-100%, 60-100%, 65-100%, 70-100%, 75-100%, 80-100%, 85-100%, 90-100%, 95-100%, 5-95%, 5-90%, 5-85%, 5-80%, 5-75%, 5-70%, 5-65%, 5-60%, 5-55%, 5-50%, 5-45%, 5-40%, 5-35%, 5-30%, 5-25%, 5-20%, 5-15%, 5-10%, 10-90%, 20-80%, 30-70%, or 40-60%.

In another aspect, provided herein is a method for treating a neurological disease in a subject in need thereof, comprising administering to the subject an effective amount of a compound of Formula (I). In some embodiments, provided herein is a method for preventing a neurological disease in a subject in need thereof, comprising administering to the subject an effective amount of a compound of Formula (I). Non-limiting examples of a neurological disease include Alzheimer's disease, multiple sclerosis (MS), amyotrophic lateral schlerosis (ALS), migraine, Bell's Palsy, ataxia, cerebral aneurysm, epilepsy, seizures, acute spinal cord injury, Guillain-Barre syndrome, meningitis, Niemann Pick disease, and Parkinson's disease. In some embodiments, the neurological disease is Alzheimer's disease or multiple sclerosis. In some embodiments, the neurological disease is Alzheimer's disease. In some embodiments, the neurological disease is multiple sclerosis.

In some embodiments, administering a compound of Formula (I) to a subject that is predisposed to a neurological disease prevents the subject from developing any symptoms of the neurological disease. In some embodiments, administering a compound of Formula (I) to a subject that is does not yet display symptoms of a neurological disease prevents the subject from developing any symptoms of the neurological disease. In some embodiments, administering a compound of Formula (I) to a subject in need thereof diminishes the extent of the neurological disease in the subject. In some embodiments, administering a compound of Formula (I) to a subject in need thereof stabilizes the neurological disease (prevents or delays the worsening of the neurological disease). In some embodiments, administering a compound of Formula (I) to a subject in need thereof delays the occurrence or recurrence of the neurological disease. In some embodiments, administering a compound of Formula (I) to a subject in need thereof slows the progression of the neurological disease. In some embodiments, administering a compound of Formula (I) to a subject in need thereof provides a partial remission of the neurological disease. In some embodiments, administering a compound of Formula (I) to a subject in need thereof provides a total remission of the neurological disease. In some embodiments, administering a compound of Formula (I) to a subject in need thereof decreases the dose of one or more other medications required to treat the neurological disease. In some embodiments, administering a compound of Formula (I) to a subject in need thereof enhances the effect of another medication used to treat the neurological disease. In some embodiments, administering a compound of Formula (I) to a subject in need thereof delays the progression of the neurological disease. In some embodiments, administering a compound of Formula (I) to a subject in need thereof increases the quality of life of the subject having a neurological disease. In some embodiments, administering a compound of Formula (I) to a subject in need thereof prolongs survival of a subject having a neurological disease.

In one aspect, provided herein is method of preventing a subject that is predisposed to a neurological disease from developing any symptoms of the neurological disease, the method comprising administering a compound of Formula (I) to the subject. In some embodiments, provided herein is a method of preventing a subject that does not yet display symptoms of a neurological disease from developing any symptoms of the neurological disease, the method comprising administering a compound of Formula (I) to the subject.

In some aspects, provided herein is a method of diminishing the extent of a neurological disease in a subject, the method comprising administering a compound of Formula (I) to the subject. In some embodiments, provided herein is a method of stabilizing a neurological disease in a subject, the method comprising administering a compound of Formula (I) to the subject. In some embodiments, the method prevents the worsening of the neurological disease. In some embodiments, the method delays the worsening of the neurological disease.

In another aspect, provided herein is a method of delaying the occurrence or recurrence of a neurological disease in a subject, the method comprising administering a compound of Formula (I) to the subject.

In some embodiments, provided herein is a method of slowing the progression of a neurological disease in a subject, the method comprising administering a compound of Formula (I) to the subject. In some embodiments, the method provides a partial remission of the neurological disease. In some embodiments, the method provides a total remission of the neurological disease.

In further aspects, provided herein is a method of decreasing the dose of one or more other medications required to treat a neurological disease in a subject, the method comprising administering a compound of Formula (I) to the subject. In some embodiments, provided herein is a method of enhancing the effect of another medication used to treat a neurological disease in a subject, the method comprising administering a compound of Formula (I) to the subject.

Also provided here is a method of delaying the progression of a neurological disease in a subject, the method comprising administering a compound of Formula (I) to the subject. In some embodiments, the method increases the quality of life of the subject having a neurological disease. In some embodiments, the method prolongs survival of the subject having a neurological disease.

In another aspect, provided herein is a method for treating neurological symptoms caused by a disease in a subject in need thereof, comprising administering to the subject an effective amount of a compound of Formula (I). In some embodiments, provided herein is a method for preventing neurological symptoms caused by a disease in a subject in need thereof, comprising administering to the subject an effective amount of a compound of Formula (I). In some embodiments, administering a compound of Formula (I) to a subject that is predisposed to a disease which causes neurological symptoms prevents the subject from developing any neurological symptoms. In some embodiments, administering a compound of Formula (I) to a subject that is does not yet display neurological symptoms of a disease which causes neurological symptoms prevents the subject from developing any neurological symptoms. In some embodiments, administering a compound of Formula (I) to a subject in need thereof diminishes the extent of the neurological symptoms caused by the disease in the subject. In some embodiments, administering a compound of Formula (I) to a subject in need thereof stabilizes the neurological symptoms of the disease (prevents or delays the worsening of the neurological symptoms). In some embodiments, administering a compound of Formula (I) to a subject in need thereof delays the occurrence or recurrence of the neurological symptoms caused by the disease. In some embodiments, administering a compound of Formula (I) to a subject in need thereof slows the progression of the neurological symptoms caused by the disease. In some embodiments, administering a compound of Formula (I) to a subject in need thereof provides a partial remission of the disease which causes neurological symptoms. In some embodiments, administering a compound of Formula (I) to a subject in need thereof provides a total remission of the disease which causes neurological symptoms. In some embodiments, administering a compound of Formula (I) to a subject in need thereof decreases the dose of one or more other medications required to treat the disease which causes neurological symptoms. In some embodiments, administering a compound of Formula (I) to a subject in need thereof enhances the effect of another medication used to treat the neurological symptoms of the disease. In some embodiments, administering a compound of Formula (I) to a subject in need thereof delays the progression of the disease which causes neurological symptoms. In some embodiments, administering a compound of Formula (I) to a subject in need thereof increases the quality of life of the subject having a disease which causes neurological symptoms. In some embodiments, administering a compound of Formula (I) to a subject in need thereof prolongs survival of a subject having a disease which causes neurological symptoms. In some embodiments, the disease is Niemann-Pick disease.

In some embodiments, compounds of Formula (I) are useful for treating a disorder selected from Alzheimer's disease, arthritis, rheumatoid arthritis, osteoarthritis, juvenile chronic arthritis, Lyme arthritis, psoriatic arthritis, reactive arthritis, and septic arthritis, spondyloarthropathy, systemic lupus erythematosus, Crohn's disease, ulcerative colitis, inflammatory bowel disease, insulin dependent diabetes mellitus, thyroiditis, asthma, allergic diseases, psoriasis, dermatitis scleroderma, graft versus host disease, organ transplant rejection (including but not limited to bone marrow and solid organ rejection), acute or chronic immune disease associated with organ transplantation, sarcoidosis, atherosclerosis, disseminated intravascular coagulation, Kawasaki's disease, Grave's disease, nephrotic syndrome, chronic fatigue syndrome, Wegener's granulomatosis, Henoch-Schoenlein purpurea, microscopic vasculitis of the kidneys, chronic active hepatitis, uveitis, septic shock, toxic shock syndrome, sepsis syndrome, cachexia, infectious diseases, parasitic diseases, acute transverse myelitis, Huntington's chorea, Parkinson's disease, stroke, primary biliary cirrhosis, hemolytic anemia, malignancies, heart failure, myocardial infarction, Addison's disease, sporadic, polyglandular deficiency type I and polyglandular deficiency type II, Schmidt's syndrome, adult (acute) respiratory distress syndrome, alopecia, alopecia areata, seronegative arthopathy, arthropathy, Reiter's disease, psoriatic arthropathy, ulcerative colitic arthropathy, enteropathic synovitis, chlamydia, yersinia and salmonella associated arthropathy, atheromatous disease/arteriosclerosis, atopic allergy, autoimmune bullous disease, pemphigus vulgaris, pemphigus foliaceus, pemphigoid, linear IgA disease, autoimmune haemolytic anaemia, Coombs positive haemolytic anaemia, acquired pernicious anaemia, juvenile pernicious anaemia, myalgic encephalitis/Royal Free Disease, chronic mucocutaneous candidiasis, giant cell arteritis, primary sclerosing hepatitis, cryptogenic autoimmune hepatitis, Acquired Immunodeficiency Disease Syndrome, Acquired Immunodeficiency Related Diseases, Hepatitis B, Hepatitis C, common varied immunodeficiency (common variable hypogammaglobulinemia), dilated cardiomyopathy, infertility, female infertility, ovarian failure, premature ovarian failure, fibrotic lung disease, chronic wound healing, cryptogenic fibrosing alveolitis, post-inflammatory interstitial lung disease, fibrosis, interstitial pneumonitis, connective tissue disease associated interstitial lung disease, mixed connective tissue disease associated lung disease, systemic sclerosis associated interstitial lung disease, rheumatoid arthritis associated interstitial lung disease, systemic lupus erythematosus associated lung disease, dermatomyositis/polytnyositis associated lung disease, Sjogren's disease associated lung disease, ankylosing spondylitis associated lung disease, vasculitic diffuse lung disease, haemosiderosis associated lung disease, drug-induced interstitial lung disease, radiation fibrosis, bronchiolitis obliterans, chronic eosinophilic pneumonia, lymphocytic infiltrative lung disease, postinfectious interstitial lung disease, gouty arthritis, autoimmune hepatitis, type-I autoimmune hepatitis (classical autoimmune or lupoid hepatitis), type-2 autoimmune hepatitis (anti-LKM antibody hepatitis), autoimmune mediated hypoglycaemia, type B insulin resistance with acanthosis nigricans, hypoparathyroidism, acute immune disease associated with organ transplantation, chronic immune disease associated with organ transplantation, osteoarthrosis, primary sclerosing cholangitis, psoriasis type 1, psoriasis type 2, idiopathic leucopaenia, autoimmune neutropaenia, renal disease NOS, glomerulonephritides, microscopic vasulitis of the kidneys, Lyme disease, discoid lupus erythematosus, male infertility idiopathic or NOS, sperm autoimmunity, multiple sclerosis (all subtypes), sympathetic ophthalmia, pulmonary hypertension secondary to connective tissue disease, Goodpasture's syndrome, pulmonary manifestation of polyarteritis nodosa, acute rheumatic fever, rheumatoid spondylitis, Still's disease, systemic sclerosis, Sjogren's syndrome, Takayasu's disease/arteritis, autoimmune thrombocytopaenia, idiopathic thrombocytopaenia, autoimmune thyroid disease, hyperthyroidism, goitrous autoimmune hypothyroidism (Hashimoto's disease), atrophic autoimmune hypothyroidism, primary myxoedema, phacogenic uveitis, primary vasculitis, vitiligo, acute liver disease, chronic liver diseases, alcoholic cirrhosis, alcohol-induced liver injury, choleosatatis, idiosyncratic liver disease, Drug-Induced hepatitis, Non-alcoholic Steatohepatitis, allergy and asthma, group B streptococci (GBS) infection, mental disorders (e.g., depression and schizophrenia), Th2 Type and ThI Type mediated diseases, acute and chronic pain (different forms of pain), and cancers such as lung, breast, stomach, bladder, colon, pancreas, ovarian, prostate and rectal cancer and hematopoietic malignancies (leukemia and lymphoma), and hematopoietic malignancies (leukemia and lymphoma), Abetalipoprotemia, Acrocyanosis, acute and chronic parasitic or infectious processes, acute leukemia, acute lymphoblastic leukemia (ALL), acute myeloid leukemia (AML), acute or chronic bacterial infection, acute pancreatitis, acute renal failure, adenocarcinomas, aerial ectopic beats, AIDS dementia complex, alcohol-induced hepatitis, allergic conjunctivitis, allergic contact dermatitis, allergic rhinitis, allograft rejection, alpha-1-antitrypsin deficiency, amyotrophic lateral sclerosis, anemia, angina pectoris, anterior horn cell degeneration, anti cd3 therapy, antiphospholipid syndrome, anti-receptor hypersensitivity reactions, aordic and peripheral aneuryisms, aortic dissection, arterial hypertension, arteriosclerosis, arteriovenous fistula, ataxia, atrial fibrillation (sustained or paroxysmal), atrial flutter, atrioventricular block, B cell lymphoma, bone graft rejection, bone marrow transplant (BMT) rejection, bundle branch block, Burkitt's lymphoma, Burns, cardiac arrhythmias, cardiac stun syndrome, cardiac tumors, cardiomyopathy, cardiopulmonary bypass inflammation response, cartilage transplant rejection, cerebellar cortical degenerations, cerebellar disorders, chaotic or multifocal atrial tachycardia, chemotherapy associated disorders, chromic myelocytic leukemia (CML), chronic alcoholism, chronic inflammatory pathologies, chronic lymphocytic leukemia (CLL), chronic obstructive pulmonary disease (COPD), chronic salicylate intoxication, colorectal carcinoma, congestive heart failure, conjunctivitis, contact dermatitis, cor pulmonale, coronary artery disease, Creutzfeldt-Jakob disease, culture negative sepsis, cystic fibrosis, cytokine therapy associated disorders, Dementia pugilistica, demyelinating diseases, dengue hemorrhagic fever, dermatitis, dermatologic conditions, diabetes, diabetes mellitus, diabetic ateriosclerotic disease, Diffuse Lewy body disease, dilated congestive cardiomyopathy, disorders of the basal ganglia, Down's Syndrome in middle age, drug-induced movement disorders induced by drugs which block CNS dopamine receptors, drug sensitivity, eczema, encephalomyelitis, endocarditis, endocrinopathy, epiglottitis, Epstein Barr virus infection, erythromelalgia, extrapyramidal and cerebellar disorders, familial hematophagocytic lymphohistiocytosis, fetal thymus implant rejection, Friedreich's ataxia, functional peripheral arterial disorders, fungal sepsis, gas gangrene, gastric ulcer, glomerular nephritis, graft rejection of any organ or tissue, gram negative sepsis, gram positive sepsis, granulomas due to intracellular organisms, hairy cell leukemia, Hallerrorden-Spatz disease, Hashimoto's thyroiditis, hay fever, heart transplant rejection, hemachromatosis, hemodialysis, hemolytic uremic syndrome/thrombolytic thrombocytopenic purpura, hemorrhage, hepatitis (A), His bundle arrythmias, HIV infection/HIV neuropathy, Hodgkin's disease, hyperkinetic movement disorders, hypersensitity reactions, hypersensitivity pneumonitis, hypertension, hypokinetic movement disorders, hypothalamic-pituitary-adrenal axis evaluation, idiopathic Addison's disease, idiopathic pulmonary fibrosis, antibody mediated cytotoxicity, Asthenia, infantile spinal muscular atrophy, inflammation of the aorta, influenza a, ionizing radiation exposure, iridocyclitis/uveitis/optic neuritis, ischemia, ischemia-reperfusion injury, ischemic stroke, juvenile rheumatoid arthritis, juvenile spinal muscular atrophy, Kaposi's sarcoma, kidney transplant rejection, legionella, leishmaniasis, leprosy, lesions of the corticospinal system, lipedema, liver transplant rejection, lymphedema, malaria, malignamt Lymphoma, malignant histiocytosis, malignant melanoma, meningitis, meningococcemia, metabolic/idiopathic, migraine headache, mitochondrial multisystem disorder, mixed connective tissue disease, monoclonal gammopathy, multiple myeloma, multiple systems degenerations (Mencel Dejerine-Thomas Shi-Drager and Machado-Joseph), myasthenia gravis, Mycobacterium avium intracellulare, Mycobacterium tuberculosis, myelodyplastic syndrome, myocardial infarction, myocardial ischemic disorders, nasopharyngeal carcinoma, neonatal chronic lung disease, nephritis, nephrosis, neurodegenerative diseases, neurogenic I muscular atrophies, neutropenic fever, non-hodgkins lymphoma, occlusion of the abdominal aorta and its branches, occulsive arterial disorders, okt3 therapy, orchitis/epidydimitis, orchitis/vasectomy reversal procedures, organomegaly, osteoporosis, pancreas transplant rejection, pancreatic carcinoma, paraneoplastic syndrome/hypercalcemia of malignancy, parathyroid transplant rejection, pelvic inflammatory disease, perennial rhinitis, pericardial disease, peripheral atherlosclerotic disease, peripheral vascular disorders, peritonitis, pernicious anemia, Pneumocystis carinii pneumonia, pneumonia, POEMS syndrome (polyneuropathy, organomegaly, endocrinopathy, monoclonal gammopathy, and skin changes syndrome), post perfusion syndrome, post pump syndrome, post-MI cardiotomy syndrome, preeclampsia, Progressive supranucleo Palsy, primary pulmonary hypertension, radiation therapy, Raynaud's phenomenon and disease, Raynaud's disease, Refsum's disease, regular narrow QRS tachycardia, renovascular hypertension, reperfusion injury, restrictive cardiomyopathy, sarcomas, scleroderma, senile chorea, Senile Dementia of Lewy body type, seronegative arthropathies, shock, sickle cell anemia, skin allograft rejection, skin changes syndrome, small bowel transplant rejection, solid tumors, specific arrythmias, spinal ataxia, spinocerebellar degenerations, streptococcal myositis, structural lesions of the cerebellum, subacute sclerosing panencephalitis, Syncope, syphilis of the cardiovascular system, systemic anaphalaxis, systemic inflammatory response syndrome, systemic onset juvenile rheumatoid arthritis, T-cell or FAB ALL, Telangiectasia, thromboangitis obliterans, thrombocytopenia, toxicity, transplants, trauma/hemorrhage, type III hypersensitivity reactions, type IV hypersensitivity, unstable angina, uremia, urosepsis, urticaria, valvular heart diseases, varicose veins, vasculitis, venous diseases, venous thrombosis, ventricular fibrillation, viral and fungal infections, vital encephalitis/aseptic meningitis, vital-associated hemaphagocytic syndrome, Wernicke-Korsakoff syndrome, Wilson's disease, xenograft rejection of any organ or tissue, acute pain, age-associated memory impairment (AAMI), anxiety attention deficit disorder, attention deficit disorder in general, attention deficit hyperactivity disorder (ADHD), bipolar disorder, cancer pain, central neuropathic pain syndromes, central post-stroke pain, chemotherapy-induced neuropathy, cognitive deficits and dysfunction in psychiatric disorders, cognitive deficits associated with aging and neurodegeneration, cognitive deficits associated with diabetes, cognitive deficits of schizophrenia, complex regional pain syndrome, declines in cognitive function in Alzheimer's and associated dementias, deficits in attention, dementia, dementia associated with Down's syndrome, dementia associated with Lewy bodies, depression in Cushing's syndrome, diminished CNS function associated with traumatic brain injury, diseases with deficits of memory, dizziness, drug abuse, epilepsy, HIV sensory neuropathy, Huntingdon's disease, hyperalgesia including neuropathic pain, inflammation and inflammatory disorders, inflammatory hyperalgesia, inflammatory pain, insulin resistance syndrome, jet lag, lack of circulation, learning, major depressive disorder, medullary thyroid carcinoma, Meniere's disease, metabolic syndrome, mild cognitive impairment, mood alteration, motion sickness, multiple sclerosis pain, narcolepsy, need for new blood vessel growth associated with vascularization of skin grafts and lack of circulation, need for new blood vessel growth associated with wound healing, neuropathic pain, neuropathy, neuropathy secondary to tumor infiltration, noninflammatory pain, obesity, obsessive compulsive disorder, painful diabetic neuropathy, panic disorder, Parkinson disease pain, pathological sleepiness, phantom limb pain, Pick's Disease, polycystic ovary syndrome, post traumatic stress disorder, post-herpetic neuralgia, post-mastectomy pain, post-surgical pain, psychotic depression, schizoaffective disorder, seizures, senile dementia, sepsis syndrome, sleep disorders, smoking cessation, spinal cord injury pain, steroid-induced acute psychosis, sub-categories of neuropathic pain including peripheral neuropathic pain syndromes, substance abuse including alcohol abuse, Syndrome X, Tourette's syndrome, treatment resistant depression, trigeminal neuralgia, type II diabetes, vertigo, and vestibular disorders.

### Pharmaceutical Compositions and Routes of Administration

The compounds provided herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions.

The compounds disclosed herein can be administered to a subject orally, topically or parenterally in the conventional form of preparations, such as capsules, microcapsules, tablets, granules, powder, troches, pills, suppositories, injections, suspensions, syrups, patches, creams, lotions, ointments, gels, sprays, solutions and emulsions. Suitable formulations can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylpyrrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous silicic acid, talc or sodium lauryl sulfate), a flavoring agent (e.g., citric acid, menthol, glycine or orange powder), a preservative (e.g, sodium benzoate, sodium bisulfite, methylparaben or propylparaben), a stabilizer (e.g., citric acid, sodium citrate or acetic acid), a suspending agent (e.g., methylcellulose, polyvinyl pyrroliclone or aluminum stearate), a dispersing agent (e.g., hydroxypropylmethylcellulose), a diluent (e.g., water), and base wax (e.g., cocoa butter, white petrolatum or polyethylene glycol). The effective amount of the compounds of Formula (I) in the pharmaceutical composition may be at a level that will exercise the desired effect.

The dose of a compound of Formula (I) to be administered to a subject is rather widely variable and can be subject to the judgment of a health-care practitioner. In any given case, the amount of the compound of Formula (I) administered will depend on such factors as the solubility of the active component, the formulation used and the route of administration.

A compound of Formula (I) can be administered orally for reasons of convenience. In one embodiment, when administered orally, a compound of Formula (I) is administered with a meal and water. In another embodiment, the compound of Formula (I) is dispersed in water or juice (e.g., apple juice or orange juice) or any other liquid and administered orally as a solution or a suspension.

The compounds disclosed herein can also be administered intradermally, intramuscularly, intraperitoneally, percutaneously, intravenously, subcutaneously, intranasally, epidurally, sublingually, intracerebrally, intravaginally, transdermally, rectally, mucosally, by inhalation, or topically to the ears, nose, eyes, or skin. The mode of administration is left to the discretion of the health-care practitioner, and can depend in-part upon the site of the medical condition.

In one embodiment, provided herein are capsules containing a compound of Formula (I) without an additional carrier, excipient or vehicle.

In another embodiment, provided herein are compositions comprising an effective amount of a compound of Formula (I) and a pharmaceutically acceptable carrier or vehicle, wherein a pharmaceutically acceptable carrier or vehicle can comprise an excipient, diluent, or a mixture thereof. In one embodiment, the composition is a pharmaceutical composition.

The compositions can be in the form of tablets, chewable tablets, capsules, solutions, parenteral solutions, troches, suppositories and suspensions and the like. Compositions can be formulated to contain a daily dose, or a convenient fraction of a daily dose, in a dosage unit, which may be a single tablet or capsule or convenient volume of a liquid. In one embodiment, the solutions are prepared from water-soluble salts, such as the hydrochloride salt. In general, all of the compositions are prepared according to known methods in pharmaceutical chemistry. Capsules can be prepared by mixing a compound of Formula (I) with a suitable carrier or diluent and filling the proper amount of the mixture in capsules. The usual carriers and diluents include, but are not limited to, inert powdered substances such as starch of many different kinds, powdered cellulose, especially crystalline and microcrystalline cellulose, sugars such as fructose, mannitol and sucrose, grain flours and similar edible powders.

Tablets can be prepared by direct compression, by wet granulation, or by dry granulation. Their formulations usually incorporate diluents, binders, lubricants and disintegrators as well as the compound. Typical diluents include, for example, various types of starch, lactose, mannitol, kaolin, calcium phosphate or sulfate, inorganic salts such as sodium chloride and powdered sugar. Powdered cellulose derivatives are also useful. Typical tablet binders are substances such as starch, gelatin and sugars such as lactose, fructose, glucose and the like. Natural and synthetic gums are also convenient, including acacia, alginates, methylcellulose, polyvinylpyrrolidine and the like. Polyethylene glycol, ethylcellulose and waxes can also serve as binders.

A lubricant might be necessary in a tablet formulation to prevent the tablet and punches from sticking in the dye. The lubricant can be chosen from such slippery solids as talc, magnesium and calcium stearate, stearic acid and hydrogenated vegetable oils. Tablet disintegrators are substances that swell when wetted to break up the tablet and release the compound. They include starches, clays, celluloses, algins and gums. More particularly, corn and potato starches, methylcellulose, agar, bentonite, wood cellulose, powdered natural sponge, cation-exchange resins, alginic acid, guar gum, citrus pulp and carboxymethyl cellulose, for example, can be used as well as sodium lauryl sulfate. Tablets can be coated with sugar as a flavor and sealant, or with film-forming protecting agents to modify the dissolution properties of the tablet. The compositions can also be formulated as chewable tablets, for example, by using substances such as mannitol in the formulation.

When it is desired to administer a compound of Formula (I) as a suppository, typical bases can be used. Cocoa butter is a traditional suppository base, which can be modified by addition of waxes to raise its melting point slightly. Water-miscible suppository bases comprising, particularly, polyethylene glycols of various molecular weights are in wide use.

The effect of the compound of Formula (I) can be delayed or prolonged by proper formulation. For example, a slowly soluble pellet of the compound of Formula (I) can be prepared and incorporated in a tablet or capsule, or as a slow-release implantable device. The technique also includes making pellets of several different dissolution rates and filling capsules with a mixture of the pellets. Tablets or capsules can be coated with a film that resists dissolution for a predictable period of time. Even the parenteral preparations can be made long-acting, by dissolving or suspending the compound of Formula (I) in oily or emulsified vehicles that allow it to disperse slowly in the serum.

### EXEMPLARY EMBODIMENTS

The present disclosure is further described by the following embodiments.

Embodiment 1. A compound of Formula (I):

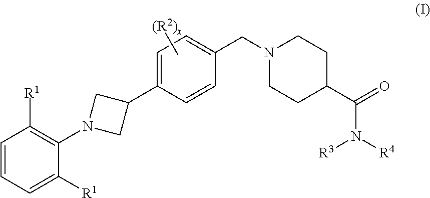

or a pharmaceutically acceptable salt thereof, wherein:
each R¹ is independently C₁-C₆ alkyl, C₃-C₆ cycloalkyl, or halo;
each R² is independently halo, C₁-C₆ alkyl, C₁-C₆ alkoxy, C₃-C₆ cycloalkyl, or C₁-C₆ haloalkyl;
x is 0-4;
R³ and R⁴ are each independently H, C₁-C₆ alkyl, C₁-C₆ alkyl-OH, —(C₁-C₆ alkylene)-O—(C₁-C₆ alkyl), C₃-C₆ cycloalkyl, —(C₁-C₆ alkylene)(C₃-C₆ cycloalkyl), or —(C₁-C₆ alkylene)NR^{5a}R^{5b}, wherein each cycloalkyl is optionally substituted by 1-5 groups selected from halo, C₁-C₆ alkyl, C₁-C₆ haloalkyl, —OH, —NR^{6a}R^{6b}, and C₁-C₆ alkoxy;
or R³ and R⁴ are taken together with the nitrogen atom to which they are attached to form a 4- to 6-membered heterocyclyl optionally substituted with 1-5 R⁷ groups, wherein the heterocyclyl optionally contains 1-2 additional heteroatoms selected from N and O;
each R^{5a} and R^{5b} is independently H or C₁-C₆ alkyl;
or R^{5a} and R^{5b} are taken together with the nitrogen atom to which they are attached to form a 4-to 6-membered heterocyclyl;
each R^{6a} and R^{6b} is independently H or C₁-C₆ alkyl; and each R⁷ is independently halo, C₁-C₆ alkyl, —OH, C₁-C₆ alkoxy, or —NR^{6a}R^{6b}.

Embodiment 2. The compound of embodiment 1, or a pharmaceutically acceptable salt thereof, wherein:
each R¹ is independently C₁-C₃ alkyl, C₃-C₆ cycloalkyl, or halo.

Embodiment 3. The compound of embodiment 1 or 2, or a pharmaceutically acceptable salt thereof, wherein:
each R¹ is independently —CH₃, cyclopropyl, F, Cl, or Br.

Embodiment 4. The compound of any one of embodiments 1-3, or a pharmaceutically acceptable salt thereof, wherein:
each R¹ is Cl.

Embodiment 5. The compound of any one of embodiments 1-4, or a pharmaceutically acceptable salt thereof, wherein:
x is 1-4; and
each R² is independently halo, C₁-C₃ alkyl, C₁-C₃ alkoxy, C₃-C₆ cycloalkyl, or C₁-C₃ haloalkyl.

Embodiment 6. The compound of embodiment 5, or a pharmaceutically acceptable salt thereof, wherein:
x is 2 or 3; and
each R² is independently Cl, F, —CH₃, —OCH₃, cyclopropyl, or —CF₃.

Embodiment 7. The compound of embodiment 6, or a pharmaceutically acceptable salt thereof, wherein:
x is 2; and
each R² is —CH₃.

Embodiment 8. The compound of any one of embodiments 1-7, or a pharmaceutically acceptable salt thereof, wherein:
R³ and R⁴ are independently H, C₁-C₅ alkyl, C₁-C₃ alkyl-OH, —(C₁-C₃ alkylene)-O—(C₁-C₃ alkyl), C₃-C₆ cycloalkyl, —(C₁-C₃ alkylene)(C₃-C₆ cycloalkyl), or —(C₁-C₃ alkylene)NR^{5a}R^{5b}, wherein each cycloalkyl is optionally substituted by 1-2 groups selected from halo, C₁-C₃ alkyl, C₁-C₃ haloalkyl, —OH, —NR^{6a}R^{6b}, and C₁-C₃ alkoxy;
each R^{5a} and R^{5b} is independently H or C₁-C₃ alkyl;
or R^{5a} and R^{5b} are taken together with the nitrogen atom to which they are attached to form a 5-to 6-membered heterocyclyl; and
each R^{6a} and R^{6b} is independently H or C₁-C₃ alkyl.

Embodiment 9. The compound of embodiment 8, or a pharmaceutically acceptable salt thereof, wherein:
R³ and R⁴ are independently H, —CH₃, —CH(CH₃)₂, —CH₂CH(CH₃)₂, —CH₂CH₂CH(CH₃)₂, —CH₂CH₂OCH₃, —CH₂CH₂OH, —CH₂CH₂N(CH₃)₂, cyclopropyl, cyclobutyl, cyclopentyl, cyclohexyl, —CH₂(cyclopropyl), —CH₂(cyclohexyl), or —CH₂CH₂(pyrrolidinyl), wherein each cycloalkyl is optionally substituted with one group selected from —OH and —N(CH₃)₂.

Embodiment 10. The compound of embodiment 9, or a pharmaceutically acceptable salt thereof, wherein:

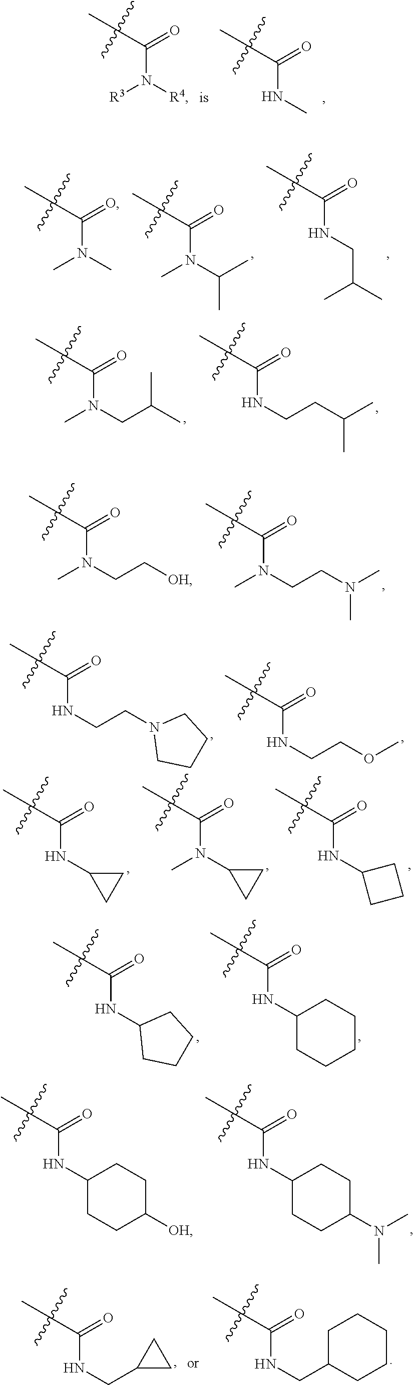

Embodiment 11. The compound of any one of embodiments 1-7, or a pharmaceutically acceptable salt thereof, wherein:
R³ and R⁴ are taken together with the nitrogen atom to which they are attached to form a 5- to 6-membered heterocyclyl optionally substituted with 1-2 R⁷ groups, wherein the heterocyclyl optionally contains one additional heteroatom selected from N and O;
each R⁷ is independently halo, C₁-C₃ alkyl, —OH, C₁-C₃ alkoxy, or —NR^{6a}R^{6b}; and
each R^{6a} and R^{6b} is independently H or C₁-C₃ alkyl.

Embodiment 12. The compound of embodiment 11, or a pharmaceutically acceptable salt thereof, wherein:
R³ and R⁴ are taken together with the nitrogen atom to which they are attached to form a 5- to 6-membered heterocyclyl optionally substituted with one R⁷ group, wherein the heterocyclyl optionally contains one additional heteroatom selected from N and O; and
R⁷ is —OH, —CH₃, —OCH₃, or —N(CH₃)₂.

Embodiment 13. The compound of embodiment 12, or a pharmaceutically acceptable salt thereof wherein:

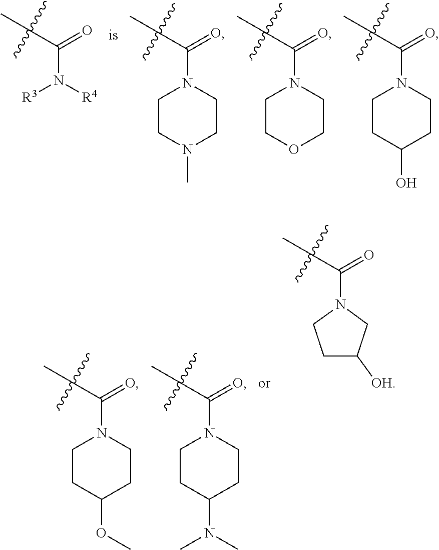

Embodiment 14. The compound of any one of embodiments 1-13, or a pharmaceutically acceptable salt thereof, wherein the compound is of Formula (II):

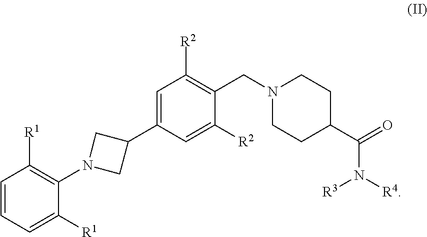

Embodiment 15. The compound of any one of embodiments 1-14, or a pharmaceutically acceptable salt thereof, wherein the compound is of Formula (IIIa):

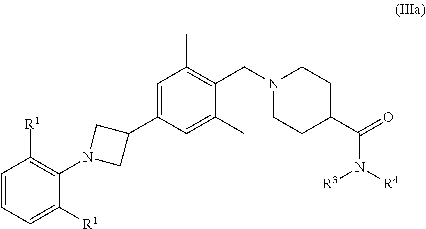

wherein:
R³ is H or C₁-C₆ alkyl;
R⁴ is H, C₁-C₆ alkyl, C₁-C₆ alkyl-OH, —(C₁-C₆ alkylene)-O—(C₁-C₆ alkyl), C₃-C₆ cycloalkyl, —(C₁-C₆ alkylene)(C₃-C₆ cycloalkyl), or —(C₁-C₆ alkylene)NR^{5a}R^{5b}, wherein each cycloalkyl is optionally substituted by 1-5 groups selected from halo, C₁-C₆ alkyl, C₁-C₆ haloalkyl, —OH, —NR^{6a}R^{6b}, and C₁-C₆ alkoxy;
R^{5a} and R^{5b} are independently H or C₁-C₆ alkyl;
or R^{5a} and R^{5b} are taken together with the nitrogen atom to which they are attached to form a 4-to 6-membered heterocyclyl; and
each R^{6a} and R^{6b} is independently H or C₁-C₆ alkyl.

Embodiment 16. The compound of any one of embodiments 1-14, or a pharmaceutically acceptable salt thereof, wherein the compound is of Formula (IVa):

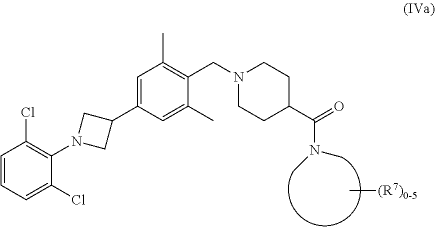

wherein

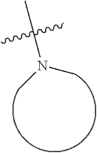

is a 4- to 6-membered heterocyclyl.

Embodiment 17. A compound selected from the compounds of Table 1 and pharmaceutically acceptable salts thereof.

Embodiment 18. A pharmaceutical composition comprising the compound of any one of embodiments 1-17, or a pharmaceutically acceptable salt thereof, and a pharmaceutically acceptable excipient.

Embodiment 19. A method of modulating sphingosine 1-phosphate receptor 5 (S1P5) comprising contacting S1P5 with an effective amount of the compound of any one of embodiments 1-17, or a pharmaceutically acceptable salt thereof, or the pharmaceutical composition of embodiment 18.

Embodiment 20. A method of treating a neurological disease in a subject in need thereof, comprising administering to the subject an effective amount of the compound of any one of embodiments 1-17, or a pharmaceutically acceptable salt thereof, or the pharmaceutical composition of embodiment 18.

Embodiment 21. The method of embodiment 20, wherein the neurological disease is Alzheimer's disease or multiple sclerosis.

### EXAMPLES

The following Examples are presented by way of illustration, not limitation. Compounds are named using the automatic name generating tool provided in ChemBiodraw Ultra (Cambridgesoft), which generates systematic names for chemical structures, with support for the Cahn-Ingold-Prelog rules for stereochemistry. One skilled in the art can modify the procedures set forth in the illustrative examples to arrive at the desired products.

Salts of the compounds described herein can be prepared by standard methods, such as inclusion of an acid (for example TFA, formic acid, or HCl) in the mobile phases during chromatography purification, or stirring of the products after chromatography purification, with a solution of an acid (for example, aqueous HCl).

The following abbreviations may be relevant for the application.

### Abbreviations

| | |
|---|---|
| AA | ammonium acetate |
| ACN | acetonitrile |
| DCM | dichloromethane |
| DMF | dimethylformamide |
| DMSO | dimethyl sulfoxide |
| EA/EtOAc | ethyl acetate |
| EDTA | ethylenediaminetetraacetic acid |
| equiv. | equivalents |
| ESI | electrospray ionization |
| h | hour(s) |
| HATU | hexafluorophosphate azabenzotriazole tetramethyl uronium |
| HEPES | 4-(2-hydroxyethyl)-1-piperazineethanesulfonic acid |
| HPLC | high performance liquid chromatography |
| LCMS | liquid chromatography mass spectrometry |
| Me-THF | methyltetrahydrofuran |
| min | minute(s) |
| MS | mass spectrometry |
| NMR | nuclear magnetic resonance |
| Pd₂(dba)₃ | tris(dibenzylideneacetone)dipalladium (0) |
| PE | petroleum ether |
| prep | preperative |
| rt | room temperature or ambient temperature |
| RT | retention time |
| TBSOTf | tert-butyldimethylsilyl trifluoromethanesulfonate |
| TFA | trifluoroacetic acid |
| THF | tetrahydrofuran |

### Synthetic Examples

### Example S1. Synthesis of 1-(4-(1-(2,6-dichlorophenyl)azetidin-3-yl)-2,6-dimethylbenzyl)piperidine-4-carboxylic acid (Acid Intermediate 1)

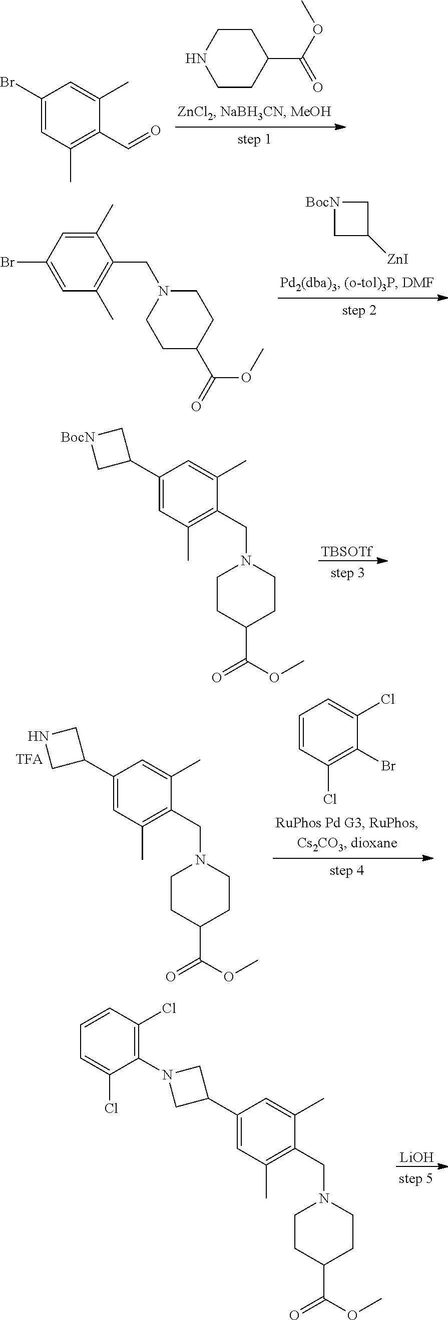

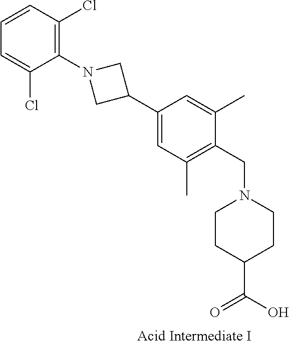 [SMILES: Cc1cc(C2CN(c3c(Cl)cccc3Cl)C2)cc(C)c1CN1CCC(C(=O)O)CC1]

### Synthesis of methyl 1-(4-bromo-2,6-dimethylbenzyl)piperidine-4-carboxylate

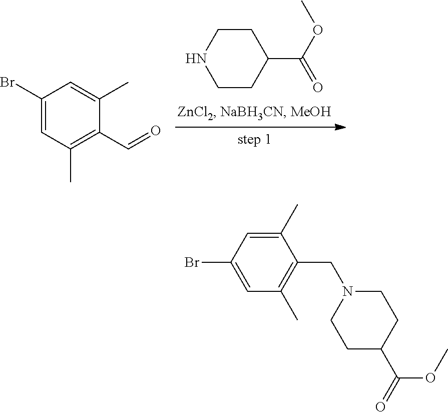

A solution of ZnCl₂ (1.9 M in Me-THF, 19.8 mL, 37.54 mmol, 2.00 equiv.) and NaBH₃CN (4.72 g, 75.09 mmol, 4.00 equiv.) in methanol (8 mL) was stirred at room temperature for 10 min. Then 4-bromo-2,6-dimethyl-benzaldehyde (4 g, 18.77 mmol, 1.00 equiv.) and methyl piperidine-4-carboxylate (5.38 g, 37.55 mmol, 2.00 equiv.) were added. The resulting mixture was stirred at 80° C. overnight. LCMS showed that the reaction was complete. The reaction mixture was poured into DCM/H₂O (1/1, 100 mL). The separated organic layer was washed with brine and dried over anhydrous Na₂SO₄ and concentrated under vacuum. The residue was purified by flash chromatography on silica gel (eluted with PE/EA, 3/1) to give methyl 1-(4-bromo-2,6-dimethylbenzyl)piperidine-4-carboxylate (3.5 g, 54.8%) as a yellow-light oil.

LCMS (ESI, m/z): 340 [M+H]⁺.

### Synthesis of methyl 1-(4-(1-(tert-butoxycarbonyl)azetidin-3-yl)-2,6-dimethylbenzyl)-piperidine-4-carboxylate

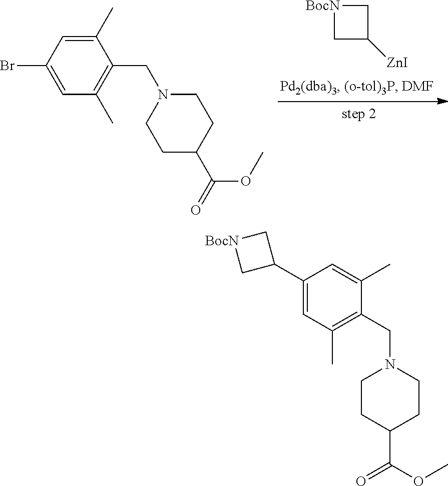

To a stirred solution of tert-butyl 3-iodoazetidine-1-carboxylate (13.24 g, 46.75 mmol, 10.0 equiv.) in DMF (75 mL) was added zinc powder (4.41 g, 67.48 mmol, 15.0 equiv.). The resulting mixture was stirred at 60° C. for 5 h. Then methyl 1-(4-bromo-2,6-dimethylbenzyl)piperidine-4-carboxylate (1.5 g, 4.41 mmol, 1.00 equiv.), Pd₂(dba)₃ (807 mg, 0.88 mmol, 0.200 equiv.) and tri(o-tolyl)phosphine (1.34 g, 4.41 mmol, 1.00 equiv.) were added. The resulting mixture was stirred at 80° C. overnight. LCMS showed that the reaction was complete. The reaction mixture was filtered through Celite, and the filtrate was concentrated under vacuum. The residue was purified by flash chromatography on C18 silica (eluted with 100% ACN) to give methyl 1-(4-(1-(tert-butoxycarbonyl)azetidin-3-yl)-2,6-dimethylbenzyl)piperidine-4-carboxylate (1.8 g, 98.0%) as a light-yellow oil.

LCMS (ESI, m/z): 417 [M+H]⁺.

### Synthesis of methyl 1-(4-(azetidin-3-yl)-2,6-dimethylbenzyl)piperidine-4-carboxylate-2,2,2-trifluoroacetaldehyde

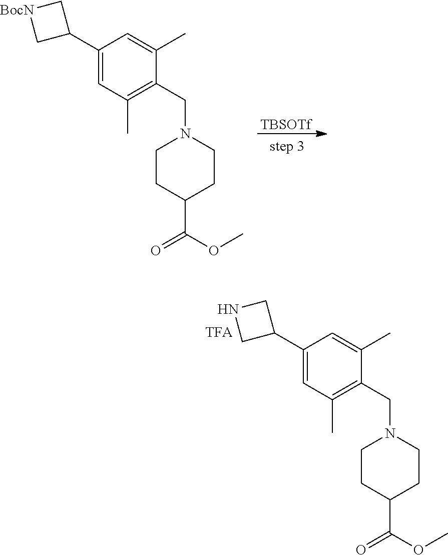 [SMILES: "TBSOTf" ; COC(=O)C1CCN(Cc2c(C)cc(C3CN(C(=O)OC(C)(C)C)C3)cc2C)CC1 ; COC(=O)C1CCN(Cc2c(C)cc(C3CNC3)cc2C)CC1]

To a stirred solution of methyl 1-(4-(1-(tert-butoxycarbonyl)azetidin-3-yl)-2,6-dimethylbenzyl)piperidine-4-carboxylate (1.8 g, 4.32 mmol, 1.00 equiv.) in DCM (2 mL) was added TBSOTf (1.53 mL, 8.64 mmol, 2.00 equiv.) dropwise at room temperature. The resulting solution was stirred at room temperature for 1 h. LCMS showed the conversion was complete. The reaction mixture was concentrated under vacuum. The residue was purified by flash chromatography on C18 silica (eluted with 22% ACN in water (0.05% TFA)) to give methyl 1-(4-(azetidin-3-yl)-2,6-dimethylbenzyl)piperidine-4-carboxylate-2,2,2-trifluoroacetaldehyde (1.3 g, 95.1%) as a light-yellow oil.

LCMS (ESI, m/z): 317 [M+H]⁺.

### Synthesis of methyl 1-(4-(1-(2,6-dichlorophenyl)azetidin-3-yl)-2,6-dimethylbenzyl)-piperidine-4-carboxylate

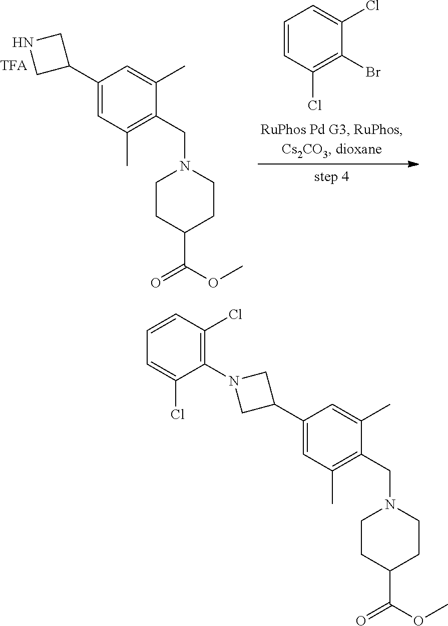

To a stirred solution of methyl 1-(4-(azetidin-3-yl)-2,6-dimethylbenzyl)piperidine-4-carboxylate-2,2,2-trifluoroacetaldehyde (2 g, 6.32 mmol, 1.00 equiv.) and 2-bromo-1,3-dichloro-benzene (2.86 g, 12.64 mmol, 2.00 equiv.) in 1,4-dioxane (2 mL) were added RuPhos Pd G3 (884 mg, 0.95 mmol, 0.15 equiv.), RuPhos (442 mg, 0.95 mmol, 0.15 equiv.), and Cs₂CO₃ (6.16 g, 18.96 mmol, 3.00 equiv.). The resulting mixture was stirred at 90° C. overnight under nitrogen atmosphere. LCMS showed that the reaction was complete. The reaction mixture was diluted with EtOAc (20 mL) and was filtered through Celite. The filtrate was concentrated under reduced pressure. The residue was purified by flash chromatography on C18 silica (eluted with 90% ACN in water (10 mM NH₄HCO₃)) to give methyl 1-(4-(1-(2,6-dichlorophenyl)azetidin-3-yl)-2,6-dimethylbenzyl)piperidine-4-carboxylate (500 mg, 17.1%) as a light-yellow oil.

LCMS (ESI, m/z): 461 [M+H]⁺.

### Synthesis of 1-(4-(1-(2,6-dichlorophenyl)azetidin-3-yl)-2,6-dimethylbenzyl)piperidine-4-carboxylic acid (Acid Intermediate I)

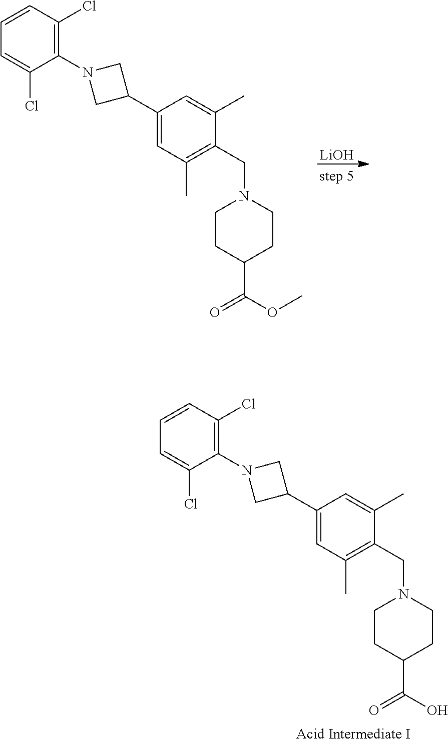 [SMILES: COC(=O)C1CCN(Cc2c(C)cc(C3CN(c4c(Cl)cccc4Cl)C3)cc2C)CC1 ; "LiOH" ; Cc1cc(C2CN(c3c(Cl)cccc3Cl)C2)cc(C)c1CN1CCC(C(=O)O)CC1]

A mixture of methyl 1-(4-(1-(2,6-dichlorophenyl)azetidin-3-yl)-2,6-dimethylbenzyl)-piperidine-4-carboxylate (25 mg, 0.54 mmol, 1.00 equiv.) and LiOH (39 mg, 1.63 mmol, 1.00 equiv.) in THE (2.5 mL) and water (2.5 mL) was stirred at room temperature overnight. LCMS showed that the reaction was complete. The reaction mixture was acidified to pH 5-6 by adding acetic acid, and then was concentrated under reduced pressure. The residue was purified by prep-HPLC (Column: XBridge Prep C18 OBD Column, 30*100 mm, 5 μm; Mobile Phase A: water (10 mmol/L NH₄HCO₃), Mobile Phase B: ACN; Flow rate: 60 mL/min; Gradient: 30% B to 60% B in 7 min; Wavelength: 254/220 nm; RT: 5.92 min) to give 1-(4-(1-(2,6-dichlorophenyl)azetidin-3-yl)-2,6-dimethylbenzyl)piperidine-4-carboxylic acid (165.1 mg, 66.7%) as a white solid.

¹H NMR (400 MHz, DMSO-d₆) δ 7.24 (d, J=7.6 Hz, 2H), 7.03 (s, 2H), 6.74 (t, J=7.6 Hz, 1H), 4.80 (t, J=8.0 Hz, 2H), 4.34 (t, J=8.0 Hz, 2H), 3.71-3.64 (m, 1H), 3.38 (s, 2H), 2.69-2.66 (m, 2H), 2.33 (s, 6H), 2.20-2.14 (m, 1H), 2.08-2.02 (m, 2H), 1.76-1.72 (m, 2H), 1.48-1.40 (m, 2H).

LCMS (ESI, m/z): 447 [M+H]+. Analytic Conditions: column: YMCMeteoric C18 BIO, 2.1*30 mm, 2.7 μm; mobile phase A: water (5 mM NH₄HCO₃), mobile phase B: acetonitrile; flow rate: 1.20 mL/min; gradient: 10% B to 95% B in 1.20 min, hold at 95% for 0.58 min, 95% B to 10% B in 0.05 min; 254 nm; RT: 0.849 min.

### Example S2. Synthesis of Amide Compounds 1-26

### General Synthesis of the Amides

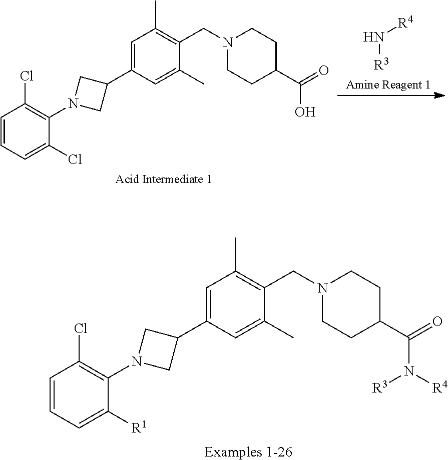

The starting material 1-(4-(1-(2,6-dichlorophenyl)azetidin-3-yl)-2,6-dimethylbenzyl)piperidine-4-carboxylic acid (Acid Intermediate I, 650 mg) was dissolved in 9.0 mL DMF (solution 1). The coupling reagent HATU (715.937 mg) was dissolved in 10.8 ml DMF (solution 2). The respective amine component (Amine Reagent I, 2.85 eq, 0.115 mmol, Table 2) was placed into a reaction vial and 1-(4-(1-(2,6-dichlorophenyl)azetidin-3-yl)-2,6-dimethylbenzyl)piperidine-4-carboxylic acid (0.250 mL of solution 1, 0.040 mmol, 1 equiv.), HATU (0.299 mL of solution 2, 0.052 mmol, 1.3 eq)) and Hunig's base (0.042 mL, 0.241 mmol, 6 equiv.) were added. The mixtures were placed into a Bohdan Miniblock XT and agitated at 400 rpm at rt for 6 hours. The crude reaction mixtures were purified via preparative reverse phase chromatography under the following conditions: Column: XBridge C18, 19 mm×200 mm, 5 μm particles; Flow Rate: 20 mL/min; Column Temperature: 25° C. Fraction collection was triggered by UV (220 nm) and MS (ESI+). Fractions containing the desired product were combined and dried via centrifugal evaporation.

Analytical Reverse Phase chromatography was used to determine the final purity.

Injection 1 conditions: Column: XBridge C18, 2.1 mm×50 mm, 1.7 m particles; Mobile Phase A: ACN/H₂O (5:95) with 0.05% TFA; Mobile Phase B: ACN/H₂O (95:5) with 0.05% TFA; Temperature: 50° C.; Gradient: 0-100% B (0.0-3.0 min), 100% B (3.0-3.5 min); Flow: 1.0 mL/min; Detection: UV (220 nm) and MS (ESI+/−).

Injection 2 conditions: Column: XBridge C18, 2.1 mm×50 mm, 1.7 μm particles; Mobile Phase A: ACN/H₂O (5:95) with 10 mM AA; Mobile Phase B: ACN/H₂O (95:5) with 10 mM AA; Temperature: 50° C.; Gradient: 0-100% B (0.0-3.0 min), 100% B (3.0-3.5 min); Flow: 1.0 mL/min; Detection: UV (220 nm) and MS (ESI+/−).

¹H NMR was acquired in deuterated DMSO.

The Amine Reagent I used in each reaction that yields Compounds 1-26 is shown in Table 2. In Table 2, the identifying number corresponding to the final compound having that same number. For example, the amine reagent associated with No. 1 in Table 2 was used to prepare Compound 1.

TABLE 2
Amine Reagent I

| No. | Structure | Name | Amount |
|---|---|---|---|
| 1 | 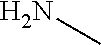 | Methylamine | 0.004 g |
| 2 | 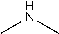 | Dimethylamine | 0.005 g |
| 3 | 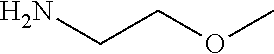 | 2-methoxyethan-1- amine | 0.009 g |
| 4 | 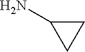 | Cyclopropylamine | 0.007 g |
| 5 | 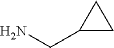 | cyclopropylmethan- amine | 0.008 g |
| 6 | 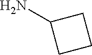 | cyclobutylamine | 0.008 g |
| 7 | 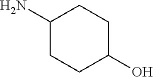 | 4-aminocyclohexan- 1-ol | 0.013 g |
| 8 | 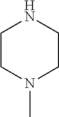 | 1-methylpiperazine | 0.012 g |
| 9 | 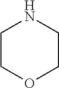 | morpholine | 0.010 g |
| 10 | 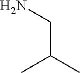 | isobutylamine | 0.008 g |
| 11 | 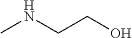 | 2-(methylamino)- ethan-1-ol | 0.009 g |
| 12 | 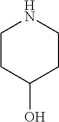 | piperidin-4-ol | 0.012 g |
| 13 | 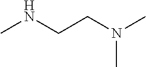 | N1,N1,N2-trimethyl- ethane-1,2-diamine | 0.012 g |
| 14 | 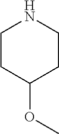 | 4-methoxypiperidine | 0.013 g |
| 15 | 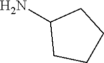 | Cyclopentylamine | 0.010 g |
| 16 | 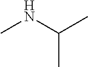 | isopropylmethylamine | 0.008 g |
| 17 | 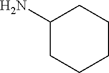 | cyclohexylamine | 0.011 g |
| 18 | 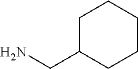 | cyclohexylmethan- amine | 0.013 g |
| 19 | 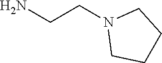 | 2-(pyrrolidin-1-yl)- ethan-1-amine | 0.013 g |
| 20 | 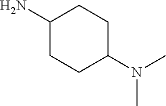 | N1,N1-dimethylcyclo- hexane-1,4-diamine | 0.016 g |
| 21 | 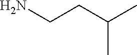 | 3-methylbutan-1-amine | 0.010 g |
| 22 | 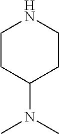 | N,N-dimethylpiperi- din-4-amine | 0.015 g |
| 23 | 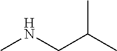 | N,2-dimethylpropan- 1-amine | 0.010 g |
| 24 | 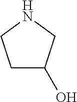 | pyrrolidin-3-ol | 0.010 g |
| 25 | 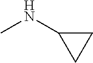 | N-methylcyclopropan- amine | 0.008 g |
| 26 | 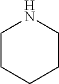 | piperidine | 0.010 g |

Characterization data of the Compounds 1-26 are shown in Table 3.

TABLE 3
Characterization data of Compounds 1-26

| Compound | MS | LC-1 | LC-2 | Purity | Yield | Yield | ¹H NMR |
|---|---|---|---|---|---|---|---|
| No. | (ESI+) | (min) | (min) | (%) | (mg) | (%) | (500 MHz, DMSO-d₆) |
| 1 | 460.1 | 1.75 | 2.46 | 93.3 | 1.2 | 6.50 | 7.65 (br, 1H), 7.24 (d, J = 7.6 |
| | | | | | | | Hz, 2H), 7.03 (s, 2H), 6.74 (t, |
| | | | | | | | J = 7.6 Hz, 1H), 4.80 (t, J = |
| | | | | | | | 8.0 Hz, 2H), 4.34 (t, J = 8.0 |
| | | | | | | | Hz, 2H), 3.70-3.64 (m, 1H), |
| | | | | | | | 3.38 (s, 2H), 2.76-2.70 (m, |
| | | | | | | | 2H), 2.54 (s, 3H), 2.33 (s, |
| | | | | | | | 6H), 2.12-1.92 (m, 3H), 1.62- |
| | | | | | | | 1.58 (m, 2H), 1.51-1.41 (m, 2H). |
| 2 | 474.2 | 1.8 | 2.61 | 97.6 | 0.7 | 3.70 | 7.24 (d, J = 7.6 Hz, 2H), 7.03 |
| | | | | | | | (s, 2H), 6.74 (t, J = 7.6 Hz, |
| | | | | | | | 1H), 4.80 (t, J = 8.0 Hz, 2H), |
| | | | | | | | 4.34 (t, J = 8.0 Hz, 2H), 3.71- |
| | | | | | | | 3.64 (m, 1H), 3.38 (s, 2H), |
| | | | | | | | 2.98 (s, 3H), 2.82 (s, 3H), |
| | | | | | | | 2.69-2.66 (m, 2H), 2.33 (s, |
| | | | | | | | 6H), 2.20-2.14 (m, 1H), 2.08- |
| | | | | | | | 2.02 (m, 2H), 1.58-1.40 (m, 4H). |
| 3 | 504.1 | 1.76 | 2.51 | 99.4 | 3 | 14.9 | 8.05 (m, 1H), 7.24 (d, J = 7.6 |
| | | | | | | | Hz, 2H), 7.03 (s, 2H), 6.75 (t, |
| | | | | | | | J = 7.6 Hz, 1H), 4.80 (t, J = |
| | | | | | | | 8.0 Hz, 2H), 4.34 (m, 3H), |
| | | | | | | | 3.70 (m, 1H), 3.60 (m, 2H), |
| | | | | | | | 3.44 (m, 4H), 3.20 (s, 3H), |
| | | | | | | | 2.72 (m, 1H), 2.51 (s, 3H), |
| | | | | | | | 2.43 (s, 3H), 2.33 (s, 3H), |
| | | | | | | | 1.90 (m, 4H). |
| 4 | 486.0 | 1.65 | 2.29 | 100 | 9 | 46.3 | 7.77 (m, 1H), 7.24 (d, J = 7.6 |
| | | | | | | | Hz, 2H), 7.03 (s, 2H), 6.74 (t, |
| | | | | | | | J = 7.6 Hz, 1H), 4.80 (t, J = |
| | | | | | | | 8.0 Hz, 2H), 4.34 (t, J = 8.0 |
| | | | | | | | Hz, 2H), 3.72 (m, 1H), 3.5 |
| | | | | | | | (m, 3H), 2.72 (m, 2H), 2.63 |
| | | | | | | | (m, 1H), 2.51 (s, 3H), 2.33 (s, |
| | | | | | | | 3H), 2.00 (m, 4H), 1.55 (m, |
| | | | | | | | 1H), 1.43 (m, 1H), 0.59 (m, |
| | | | | | | | 2H), 0.32 (m, 2H). |
| 5 | 500.1 | 1.86 | 2.72 | 100 | 5.8 | 29 | 7.68 (br, 1H), 7.23 (d, J = 7.6 |
| | | | | | | | Hz, 2H), 7.03 (s, 2H), 6.75 (t, |
| | | | | | | | J = 7.6 Hz, 1H), 4.80 (t, J = |
| | | | | | | | 8.0 Hz, 2H), 4.33 (t, J = 8.0 |
| | | | | | | | Hz, 2H), 3.71-3.64 (m, 1H), |
| | | | | | | | 3.38 (s, 2H), 2.86-2.83 (m, |
| | | | | | | | 2H), 2.74-2.72 (m, 2H), 2.32 |
| | | | | | | | (s, 6H), 2.16-2.10 (m, 1H), |
| | | | | | | | 2.06-2.00 (m, 2H), 1.69-1.62 |
| | | | | | | | (m, 1H) 1.59-1.55 (m, 2H), |
| | | | | | | | 1.42-1.34 (m, 2H), 0.58 (d, 4H). |
| 6 | 500.1 | 1.99 | 2.84 | 99.3 | 10.7 | 53.5 | 7.94 (m, 1H), 7.24 (d, J = 7.6 |
| | | | | | | | Hz, 2H), 7.03 (s, 2H), 6.75 (t, |
| | | | | | | | J = 7.6 Hz, 1H), 4.80 (t, J = |
| | | | | | | | 8.0 Hz, 2H), 4.34 (t, J = 8.0 |
| | | | | | | | Hz, 2H), 4.15 (m, 2H), 3.70 |
| | | | | | | | (m, 1H), 3.44 (m, 3H), 3.35 |
| | | | | | | | (m, 1H), 2.72 (m, 1H), 2.63 |
| | | | | | | | (m, 1H), 2.51 (s, 3H), 2.41 |
| | | | | | | | (m, 2H), 2.33 (s, 3H), 2.20- |
| | | | | | | | 1.65 (series of m, 8H). |
| 7 | 544.2 | 1.83 | 2.51 | 100 | 2.0 | 9.19 | 7.80 (br, 1H), 7.24 (m, 2H), |
| | | | | | | | 7.04 (s, 2H), 6.76 (t, J = 7.6 |
| | | | | | | | Hz, 1H), 4.80 (br, 2H), 4.30 |
| | | | | | | | (br, 2H), 3.75-3.68 (m, 1H), |
| | | | | | | | 3.65-3.60 (m, 1H), 3.42 (s, |
| | | | | | | | 2H), 3.18 (br, 1H), 2.69-2.66 |
| | | | | | | | (m, 2H), 2.33 (s, 6H), 2.20- |
| | | | | | | | 2.14 (m, 1H), 2.08-2.02 (m, |
| | | | | | | | 2H), 1.90-1.80 (m, 4H), 1.78- |
| | | | | | | | 1.74 (m, 2H), 1.70-1.60 (m, |
| | | | | | | | 2H), 1.55-1.45 (m, 2H), 1.22- |
| | | | | | | | 1.12 (m, 2H). |
| 8 | 529.1 | 1.41 | 2.38 | 100 | 18.2 | 86 | 7.24 (m, 2H), 7.04 (s, 2H), |
| | | | | | | | 6.74 (t, J = 7.6 Hz, 1H), 4.80 |
| | | | | | | | (t, J = 8.0 Hz, 2H), 4.33 (t, J = |
| | | | | | | | 8.0 Hz, 2H), 3.74-3.68 (m, |
| | | | | | | | 1H), 3.50-3.40 (m, 2H), 2.78- |
| | | | | | | | 2.70 (m, 1H), 2.55 (s, 2H), |
| | | | | | | | 2.50 (s, 6H), 2.35 (s, 6H), |
| | | | | | | | 2.34-2.28 (m, 2H), 2.26-2.20 |
| | | | | | | | (m, 3H), 2.05-1.95 (m, 2H), |
| | | | | | | | 1.55-1.40 (m, 4H). |
| 9 | 516.1 | 1.65 | 2.3 | 100 | 16 | 77.5 | 7.24 (d, J = 7.6 Hz, 2H), 7.03 |
| | | | | | | | (s, 2H), 6.74 (t, J = 7.6 Hz, |
| | | | | | | | 1H), 4.80 (t, J = 8.0 Hz, 2H), |
| | | | | | | | 4.34 (t, J = 8.0 Hz, 2H), 3.71- |
| | | | | | | | 3.64 (m, 2H), 3.48 (m, 6H), |
| | | | | | | | 2.69-2.66 (m, 2H), 2.60 (m, |
| | | | | | | | 2H), 2.50 (s, 3H), 2.33 (s, |
| | | | | | | | 4H), 2.08 (m, 1H), 1.48-1.40 |
| | | | | | | | (m, 6H). |
| 10 | 502.1 | 2.0 | 2.0 | 100 | 10.1 | 50.3 | 7.70 (br, 1H), 7.23 (d, J = 7.6 |
| | | | | | | | Hz, 2H), 7.03 (s, 2H), 6.75 (t, |
| | | | | | | | J = 7.6 Hz, 1H), 4.80 (t, J = |
| | | | | | | | 8.0 Hz, 2H), 4.33 (t, J = 8.0 |
| | | | | | | | Hz, 2H), 3.71-3.64 (m, 1H), |
| | | | | | | | 3.38 (s, 2H), 2.86-2.83 (m, |
| | | | | | | | 2H), 2.74-2.72 (m, 2H), 2.32 |
| | | | | | | | (s, 6H), 2.16-2.10 (m, 1H), |
| | | | | | | | 2.06-2.00 (m, 2H), 1.69-1.62 |
| | | | | | | | (m, 1H) 1.59-1.55 (m, 2H), |
| | | | | | | | 1.50-1.42 (m, 2H), 0.80 (d, 6H). |
| 11 | 504.1 | 1.79 | 2.1 | 100 | 15.7 | 77.9 | 7.24 (d, J = 7.6 Hz, 2H), 7.03 |
| | | | | | | | (s, 2H), 6.75 (t, J = 7.6 Hz, |
| | | | | | | | 1H), 4.80 (t, J = 8.0 Hz, 2H), |
| | | | | | | | 4.65 (m, 1H), 4.31 (t, J = 8.0 |
| | | | | | | | Hz, 2H), 3.65 (m, 1H), 3.60 |
| | | | | | | | (m, 2H), 3.44 (m, 4H), 3.10 |
| | | | | | | | (s, 3H), 2.72 (m, 2H), 2.63 |
| | | | | | | | (m, 1H), 2.51 (s, 3H), 2.30 (s, |
| | | | | | | | 3H), 2.05 (m, 2H), 1.55 (m, 4H). |
| 12 | 530.1 | 1.66 | 2.5 | 99.3 | 20.6 | 97 | 7.24 (d, J = 7.6 Hz, 2H), 7.03 |
| | | | | | | | (s, 2H), 6.74 (t, J = 7.6 Hz, |
| | | | | | | | 1H), 4.80 (t, J = 8.0 Hz, 2H), |
| | | | | | | | 4.34 (t, J = 8.0 Hz, 2H), 3.80- |
| | | | | | | | 2.70 (series of m, 6H), 3.22 |
| | | | | | | | (s, 3H), 2.73, (m, 2H), 2.51 |
| | | | | | | | (m, 1H), 2.50 (s, 3H), 2.35 (s, |
| | | | | | | | 3H), 2.20-2.14 (m, 1H), 2.05 |
| | | | | | | | (m, 2H), 1.76-1.20 (series of |
| | | | | | | | m, 6H). |
| 13 | 531.1 | 1.43 | 2.3 | 100 | 15.3 | 72 | 7.24 (d, J = 7.6 Hz, 2H), 7.03 |
| | | | | | | | (s, 2H), 6.74 (t, J = 7.6 Hz, |
| | | | | | | | 1H), 4.80 (t, J = 8.0 Hz, 2H), |
| | | | | | | | 4.34 (t, J = 8.0 Hz, 2H), 3.71- |
| | | | | | | | 3.55 (m, 3H), 3.38 (m, 3H), |
| | | | | | | | 3.00 (3, 3H), 2.75 (m, 2H), |
| | | | | | | | 2.53 (m, 4H), 2.40 (m, 1H), |
| | | | | | | | 2.33 (m, 4H), 2.25 (s, 3H), |
| | | | | | | | 2.08 (m, 1H), 1.90 (s, 3H), |
| | | | | | | | 1.68-1.40 (m, 4H). |
| 14 | 544.0 | 1.69 | 2.4 | 100 | 18.2 | 83.6 | 7.24 (d, J = 7.6 Hz, 2H), 7.03 |
| | | | | | | | (s, 2H), 6.74 (t, J = 7.6 Hz, |
| | | | | | | | 1H), 4.80 (t, J = 8.0 Hz, 2H), |
| | | | | | | | 4.34 (t, J = 8.0 Hz, 2H), 3.80- |
| | | | | | | | 2.70 (series of m, 6H), 3.22 |
| | | | | | | | (s, 3H), 2.73, (m, 2H), 2.51 |
| | | | | | | | (m, 1H), 2.50 (s, 3H), 2.48 (s, |
| | | | | | | | 3H), 2.35 (s, 3H), 2.20-2.14 |
| | | | | | | | (m, 1H), 2.05 (m, 2H), 1.76- |
| | | | | | | | 1.20 (series of m, 6H). |
| 15 | 514.1 | 2.03 | 2.89 | 97.9 | 4.9 | 23.8 | 7.92 (br, 1H), 7.23 (m, 2H), |
| | | | | | | | 7.08 (s, 2H), 6.76 (t, J = 7.6 |
| | | | | | | | Hz, 1H), 4.80 (t, J = 8.0 Hz, |
| | | | | | | | 2H), 4.32 (t, J = 8.0 Hz, 2H), |
| | | | | | | | 3.75-3.68 (m, 1H), 3.65-3.60 |
| | | | | | | | (m, 1H), 3.42 (s, 2H), 2.69- |
| | | | | | | | 2.66 (m, 2H), 2.33 (s, 6H), |
| | | | | | | | 2.20-2.14 (m, 1H), 2.08-2.02 |
| | | | | | | | (m, 2H), 1.90-1.80 (m, 4H), |
| | | | | | | | 1.78-1.74 (m, 2H), 1.70-1.60 |
| | | | | | | | (m, 2H), 1.55-1.45 (m, 2H), |
| | | | | | | | 1.40-1.30 (m, 2H). |
| 16 | 502.0 | 1.74 | 2.48 | 100 | 16.4 | 81.7 | 7.24 (d, J = 7.6 Hz, 2H), 7.03 |
| | | | | | | | (s, 2H), 6.74 (t, J = 7.6 Hz, |
| | | | | | | | 1H), 4.84 (d, J = 7.6 Hz, 2H), |
| | | | | | | | 4.65 (m, 1H), 4.31 (d, J = 7.6 |
| | | | | | | | Hz, 2H), 4.15 (m, 1H), 3.70, |
| | | | | | | | (m, 2H), 2.88 (s, 3H), 2.73, |
| | | | | | | | (m, 2H), 2.50 (s, 3H), 2.38 (s, |
| | | | | | | | 3H), 2.06 (m, 2H), 1.46 (m, |
| | | | | | | | 4H), 1.13 (d, J = 6.5 HZ, 3H), |
| | | | | | | | 0.97 (d, J = 6.5 Hz, 3H). |
| 17 | 528.0 | 2.12 | 3.03 | 100 | 2.2 | 10.4 | 7.60 (br, 1H), 7.24 (m, 2H), |
| | | | | | | | 7.04 (s, 2H), 6.76 (t, J = 7.6 |
| | | | | | | | Hz, 1H), 4.80 (t, J = 8.0 Hz, |
| | | | | | | | 2H), 4.30 (t, J = 8.0 Hz, 2H), |
| | | | | | | | 3.75-3.68 (m, 1H), 3.65-3.60 |
| | | | | | | | (m, 1H), 3.42 (s, 2H), 2.69- |
| | | | | | | | 2.66 (m, 2H), 2.33 (s, 6H), |
| | | | | | | | 2.20-2.14 (m, 1H), 2.08-2.02 |
| | | | | | | | (m, 2H), 1.90-1.80 (m, 4H), |
| | | | | | | | 1.78-1.74 (m, 2H), 1.70-1.60 |
| | | | | | | | (m, 2H), 1.55-1.45 (m, 2H), |
| | | | | | | | 1.30-1.20 (m, 2H), 1.12-1.08 |
| | | | | | | | (m, 2H). |
| 18 | 542.1 | 2.09 | 3.04 | 99.4 | 3.1 | 14.3 | 7.69 (br, 1H), 7.24 (m, 2H), |
| | | | | | | | 7.04 (s, 2H), 6.78 (t, J = 7.6 |
| | | | | | | | Hz, 1H), 4.80 (t, J = 8.0 Hz, |
| | | | | | | | 2H), 4.30 (t, J = 8.0 Hz, 2H), |
| | | | | | | | 3.75-3.68 (m, 1H), 3.65-3.60 |
| | | | | | | | (m, 1H), 3.42 (s, 2H), 3.18 |
| | | | | | | | (br, 2H), 2.69-2.66 (m, 2H), |
| | | | | | | | 2.33 (s, 6H), 2.20-2.14 (m, |
| | | | | | | | 1H), 2.08-2.02 (m, 2H), 1.90- |
| | | | | | | | 1.80 (m, 4H), 1.78-1.74 (m, |
| | | | | | | | 2H), 1.70-1.60 (m, 2H), 1.55- |
| | | | | | | | 1.45 (m, 2H), 1.20-1.11 (m, |
| | | | | | | | 2H), 0.90-0.80 (m, 2H). |
| 19 | 543.2 | 1.63 | 2.28 | 100 | 0.7 | 3.22 | 7.52 (br, 1H), 7.02 (m, 2H), |
| | | | | | | | 6.82 (s, 2H), 65.2 (t, J = 7.6 |
| | | | | | | | Hz, 1H), 4.59 (t, J = 8.0 Hz, |
| | | | | | | | 2H), 4.11 (t, J = 8.0 Hz, 2H), |
| | | | | | | | 3.48-3.43 (m, 1H), 2.94-2.90 |
| | | | | | | | (m, 1H), 2.52-2.48 (m, 2 H), |
| | | | | | | | 2.40 (s, 6H), 2.10 (br, 2H), |
| | | | | | | | 1.90-1.85 (m, 2H), 1.90-1.80 |
| | | | | | | | (m, 4H), 1.78-1.72 (m, 2H), |
| | | | | | | | 1.70-1.60 (m, 2H), 1.50-1.45 |
| | | | | | | | (m, 4H), 1.39-1.34 (m, 2H), |
| | | | | | | | 1.25-1.20 (m, 2H). |
| 20 | 571.1 | 1.45 | 2.17 | 99.5 | 17 | 74.4 | 7.60 (br, 1H), 7.24 (m, 2H), |
| | | | | | | | 7.04 (s, 2H), 6.76 (t, J = 7.6 |
| | | | | | | | Hz, 1H), 4.80 (t, J = 8.0 Hz, |
| | | | | | | | 2H), 4.33 (t, J = 8.0 Hz, 2H), |
| | | | | | | | 3.74-3.68 (m, 1H), 2.78-2.70 |
| | | | | | | | (m, 1H), 2.55 (s, 2H), 2.50 (s, |
| | | | | | | | 6H), 2.35 (s, 6H), 2.26-2.20 |
| | | | | | | | (2H), 2.05-1.95 (m, 2H), 1.78- |
| | | | | | | | 1.74 (m, 2H), 1.65-1.58 (m, |
| | | | | | | | 4H), 1.55-1.40 (m, 4H), 1.30- |
| | | | | | | | 1.22 (m, 2H), 1.20-1.10 (m, 2H). |
| 21 | 516.1 | 1.99 | 2.92 | 100 | 9.7 | 47 | 7.68 (br, 1H), 7.24 (d, J = 7.6 |
| | | | | | | | Hz, 2H), 7.03 (s, 2H), 6.74 (t, |
| | | | | | | | J = 7.6 Hz, 1H), 4.80 (t, J = |
| | | | | | | | 8.0 Hz, 2H), 4.34 (m, J = 8.0 |
| | | | | | | | Hz, 2H), 3.71-3.64 (m, 1H), |
| | | | | | | | 3.38 (s, 2H), 3.05-2.99 (m, |
| | | | | | | | 2H), 2.69-2.66 (m, 2H), 2.33 |
| | | | | | | | (s, 6H), 2.20-2.14 (m, 1H), |
| | | | | | | | 2.08-2.02 (m, 2H), 1.98-1.90 |
| | | | | | | | (m, 2H), 1.58-1.40 (m, 3H), |
| | | | | | | | 1.27 (m, 2H), 0.88 (d, 6H). |
| 22 | 557.2 | 1.71 | 2.36 | 100 | 20.1 | 90.2 | 7.02 (d, 2H), 6.82 (s, 2H), |
| | | | | | | | 6.52 (t, J = 7.6 Hz, 1H), 4.59 |
| | | | | | | | (t, J = 8.0 Hz, 2H), 4.11 (t, J = |
| | | | | | | | 8.0 Hz, 2H), 3.48-3.43 (m, |
| | | | | | | | 1H), 2.94-2.90 (m, 1H), 2.52- |
| | | | | | | | 2.48 (m, 2 H), 2.40 (s, 6H), |
| | | | | | | | 2.10 (br, 2H), 1.90-1.85 (m, |
| | | | | | | | 2H), 1.90-1.80 (m, 4H), 1.78- |
| | | | | | | | 1.72 (m, 2H), 1.70-1.60 (m, |
| | | | | | | | 2H), 1.50-1.45 (m, 4H), 1.39- |
| | | | | | | | 1.34 (m, 2H), 1.25-1.20 (m, 2H). |
| 23 | 516.0 | 1.81 | 2.61 | 100 | 11 | 53.3 | 7.24 (d, J = 7.6 Hz, 2H), 7.03 |
| | | | | | | | (s, 2H), 6.74 (t, J = 7.6 Hz, |
| | | | | | | | 1H), 4.84 (d, J = 7.6 Hz, 2H), |
| | | | | | | | 4.31 (d, J = 7.6 Hz, 2H), 3.68, |
| | | | | | | | (m, 2H), 3.11 (m, 2H), 2.78, |
| | | | | | | | (s, 3H), 2.72, (s, 2H), 2.50 (s, |
| | | | | | | | 3H), 2.33 (s, 3H), 2.06 (m, |
| | | | | | | | 1H), 1.87 (m, 2H), 1.52 (m, |
| | | | | | | | 5H), 0.88 (d, J = 6.5 HZ, 3H), |
| | | | | | | | 0.75 (d, J = 6.5 Hz, 3H). |
| 24 | 516.1 | 1.58 | 2.1 | 100 | 16.1 | 78 | 7.24 (d, J = 7.6 Hz, 2H), 7.03 |
| | | | | | | | (s, 2H), 6.74 (t, J = 7.6 Hz, |
| | | | | | | | 1H), 4.80 (t, J = 8.0 Hz, 2H), |
| | | | | | | | 4.34 (t, J = 8.0 Hz, 2H), 3.80- |
| | | | | | | | 2.70 (series of m, 6H), 3.22 |
| | | | | | | | (s, 3H), 2.73, (m, 2H), 2.51 |
| | | | | | | | (m, 1H), 2.50 (s, 3H), 2.48 (s, |
| | | | | | | | 3H), 2.35 (s, 3H), 2.20-2.14 |
| | | | | | | | (m, 1H), 2.05 (m, 2H), 1.76- |
| | | | | | | | 1.20 (series of m, 6H). |
| 25 | 500.1 | 1.72 | 2.9 | 92.4 | 1.3 | 6.5 | 7.24 (m, 2H), 7.04 (s, 2H), |
| | | | | | | | 6.74 (t, J = 7.6 Hz, 1H), 4.80 |
| | | | | | | | (t, J = 8.0 Hz, 2H), 4.33 (t, J = |
| | | | | | | | 8.0 Hz, 2H), 3.74-3.68 (m, |
| | | | | | | | 1H), 3.50-3.40 (m, 2H), 2.78- |
| | | | | | | | 2.70 (m, 1H), 2.55 (s, 2H), |
| | | | | | | | 2.75 (s, 3H), 2.35 (s, 6H), |
| | | | | | | | 2.10-2.01 (m, 2H), 1.91 (br, |
| | | | | | | | 1H) 1.65-1.40 (m, 4H), 0.83 |
| | | | | | | | (br, 2H), 0.72 (br, 2H). |
| 26 | 514.1 | 1.94 | 2.91 | 100 | 5 | 24.3 | 7.24 (d, J = 7.6 Hz, 2H), 7.03 |
| | | | | | | | (s, 2H), 6.74 (t, J = 7.6 Hz, |
| | | | | | | | 1H), 4.80 (t, J = 8.0 Hz, 2H), |
| | | | | | | | 4.34 (t, J = 8.0 Hz, 2H), 3.70- |
| | | | | | | | 3.64 (m, 1H), 3.40-3.35 (br, |
| | | | | | | | 2H), 2.76-2.68 (br, 2H), 2.33 |
| | | | | | | | (s, 6H), 2.12-2.02 (m, 2H), |
| | | | | | | | 1.60-1.35 (m, 15H). |

### Biological Examples

### Example B1. Cell Membrane Preparations

CHO cells expressing recombinant S1P5 receptors were cultured in 500 cm² culture trays and, once confluent, rinsed and detached with cell-lifting buffer (10 mM HEPES, 154 mM NaCl, 6.85 mM EDTA, pH 7.4). Cells were then pelleted by centrifugation, resuspended, and homogenized in membrane preparation buffer (10 mM HEPES and 10 mM EDTA, pH 7.4) using a Polytron PT 1200E homogenizer (Kinematica, Luzern, Switzerland). Cellular proteins were pelleted by centrifugation at 48,000×g at 4° C. for 30 minutes. The resulting supernatant was discarded, and the pellet was re-suspended again in membrane preparation buffer, homogenized for a second time, and then centrifuged again as described above. The final cellular protein pellet was suspended in ice cold resuspension buffer (10 mM HEPES and 0.1 mM EDTA, pH 7.4), divided into aliquots, and stored at −80° C. until use.

### Example B2. GTPγS Binding Assay

Functional binding assays for [³⁵S]-GTPγS were performed in 96-well non-binding surface plates with a final volume of 200 μL. The test compounds were serially diluted in DMSO and added to assay plates using a Tecan D300E digital printer with a total volume of 0.4 μL. The control sphingosine-1-phosphate (S1P) was prepared separately by preparing a 400 μM stock solution from a 100 nmol pellet of SIP in 10 mM Na₂CO₃ with 2% β-cyclodextrin. The serial dilution of S1P was done using complete assay buffer (20 mM HEPES, 10 mM MgCl₂, 100 mM NaCl, 1 mM EDTA, 0.1% fatty acid free bovine serum albumin (BSA), and 30 μg/mL saponin, pH 7.4) and transferred to wells already containing 0.4 μL DMSO. All the wells were then loaded to a total volume of 40 μL of complete assay buffer, except the non-specific binding (NSB) wells. For NSB wells, 40 μL/well of 50 μM GTPγS (Sigma Aldrich, cat #G8634, St. Louis, MO) was added to wells containing 0.4 μL of DMSO. The assay was started by the addition of 120 μL/well of CHO—S1P receptor membrane solution containing 40 μg/mL of membrane protein, 16.67 μM guanosine diphosphate (GDP; Sigma Aldrich, cat #G7127, St. Louis, MO), and 2.5 mg/mL of WGA PVT SPA beads in complete buffer. Assay plates were then sealed and incubated at room temperature with gentle agitation for 30 minutes. Next, 40 μL/well of 1 nM of [³⁵S]-GTPγS (PerkinElmer, cat #NEG030X250UC, Waltham, MA) in basic assay buffer (20 mM HEPES, 10 mM MgCl₂, 100 mM NaCl, and 1 mM EDTA, pH7.4) was added to the assay plates to yield a final concentration of 200 pM and the plates were further incubated for 40 minutes at room temperature with gentle agitation. The assay was terminated by centrifugation of the plates at 1000 rpm for 3 minutes using an Eppendorf 5810R centrifuge (Eppendorf, Hamburg, Germany) and G protein bound radioactivity was quantitated using a MicroBeta2 microplate scintillation counter (PerkinElmer, Waltham, MA). As G protein bound radioactivity directly correlates to receptor activation and coupling to the G protein, this assay is a measure of S1P5 agonism. Results are shown in Table 4.

TABLE 4
S1P5 GTPγS Binding of Exemplary Compounds.

| Compound No. | Activity (nM) |
|---|---|
| 1 | ++++ |
| 2 | ++++ |
| 3 | ++++ |
| 4 | +++ |
| 5 | +++ |
| 6 | +++ |
| 7 | ++ |
| 8 | ++ |
| 9 | ++ |
| 10 | ++ |
| 11 | ++ |
| 12 | ++ |
| 13 | ++ |
| 14 | ++ |
| 15 | + |
| 16 | + |
| 17 | + |
| 18 | + |
| 19 | + |
| 20 | + |
| 21 | + |
| 22 | + |
| 23 | + |
| 24 | + |
| 25 | + |
| 26 | + |

++++ indicates binding between greater than 1 nM and ≤10 nM
+++ indicates binding between greater than 10 nM and ≤100 nM
++ indicates binding between greater than 100 nM and ≤1,000 nM
+ indicates binding between greater than 1,000 nM and ≤10,000 nM

Although the present invention has been described in some detail by way of illustration and example for purposes of clarity of understanding, the descriptions and examples should not be construed as limiting the scope of the invention. The disclosures of all patent and scientific literature cited herein are expressly incorporated herein in their entirety by reference.

1. A compound of Formula (I): 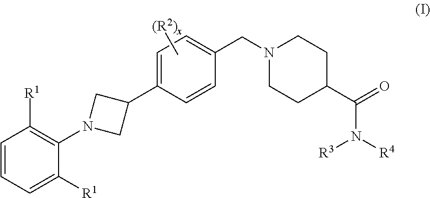
or a pharmaceutically acceptable salt thereof, wherein:
each R¹ is independently C₁-C₆ alkyl, C₃-C₆ cycloalkyl, or halo;
each R² is independently halo, C₁-C₆ alkyl, C₁-C₆ alkoxy, C₃-C₆ cycloalkyl, or C₁-C₆ haloalkyl;
x is 0-4;
R³ and R⁴ are each independently H, C₁-C₆ alkyl, C₁-C₆ alkyl-OH, —(C₁-C₆ alkylene)-O—(C₁-C₆ alkyl), C₃-C₆ cycloalkyl, —(C₁-C₆ alkylene)(C₃-C₆ cycloalkyl), or —(C₁-C₆ alkylene)NR^{5a}R^{5b}, wherein each cycloalkyl is optionally substituted by 1-5 groups selected from halo, C₁-C₆ alkyl, C₁-C₆ haloalkyl, —OH, —NR^{6a}R^{6b}, and C₁-C₆ alkoxy;
or R³ and R⁴ are taken together with the nitrogen atom to which they are attached to form a 4- to 6-membered heterocyclyl optionally substituted with 1-5 R⁷ groups, wherein the heterocyclyl optionally contains 1-2 additional heteroatoms selected from N and O;
each R^{5a} and R^{5b} is independently H or C₁-C₆ alkyl;
or R^{5a} and R^{5b} are taken together with the nitrogen atom to which they are attached to form a 4-to 6-membered heterocyclyl;
each R^{6a} and R^{6b} is independently H or C₁-C₆ alkyl; and
each R⁷ is independently halo, C₁-C₆ alkyl, —OH, C₁-C₆ alkoxy, or —NR^{6a}R^{6b}.

2. The compound of claim 1, or a pharmaceutically acceptable salt thereof, wherein:
each R¹ is independently C₁-C₃ alkyl, C₃-C₆ cycloalkyl, or halo.

3. The compound of claim 1 or 2, or a pharmaceutically acceptable salt thereof, wherein:
each R¹ is independently —CH₃, cyclopropyl, F, Cl, or Br.

4. The compound of any one of claims 1-3, or a pharmaceutically acceptable salt thereof,
wherein:
each R¹ is Cl.

5. The compound of any one of claims 1-4, or a pharmaceutically acceptable salt thereof,
wherein:
x is 1-4; and
each R² is independently halo, C₁-C₃ alkyl, C₁-C₃ alkoxy, C₃-C₆ cycloalkyl, or C₁-C₃ haloalkyl.

6. The compound of claim 5, or a pharmaceutically acceptable salt thereof, wherein:
x is 2 or 3; and
each R² is independently Cl, F, —CH₃, —OCH₃, cyclopropyl, or —CF₃.

7. The compound of claim 6, or a pharmaceutically acceptable salt thereof, wherein:
x is 2; and
each R² is —CH₃.

8. The compound of any one of claims 1-7, or a pharmaceutically acceptable salt thereof,
wherein:
R³ and R⁴ are independently H, C₁-C₅ alkyl, C₁-C₃ alkyl-OH, —(C₁-C₃ alkylene)-O—(C₁-C₃ alkyl), C₃-C₆ cycloalkyl, —(C₁-C₃ alkylene)(C₃-C₆ cycloalkyl), or —(C₁-C₃ alkylene)NR^{5a}R^{5b}, wherein each cycloalkyl is optionally substituted by 1-2 groups selected from halo, C₁-C₃ alkyl, C₁-C₃ haloalkyl, —OH, —NR^{6a}R^{6b}, and C₁-C₃ alkoxy;
each R^{5a} and R^{5b} is independently H or C₁-C₃ alkyl;
or R^{5a} and R^{5b} are taken together with the nitrogen atom to which they are attached to form a 5-to 6-membered heterocyclyl; and
each R^{6a} and R^{6b} is independently H or C₁-C₃ alkyl.

9. The compound of claim 8, or a pharmaceutically acceptable salt thereof, wherein:
R³ and R⁴ are independently H, —CH₃, —CH(CH₃)₂, —CH₂CH(CH₃)₂, —CH₂CH₂CH(CH₃)₂, —CH₂CH₂OCH₃, —CH₂CH₂OH, —CH₂CH₂N(CH₃)₂, cyclopropyl, cyclobutyl, cyclopentyl, cyclohexyl, —CH₂(cyclopropyl), —CH₂(cyclohexyl), or —CH₂CH₂(pyrrolidinyl), wherein each cycloalkyl is optionally substituted with one group selected from —OH and —N(CH₃)₂.

10. The compound of claim 9, or a pharmaceutically acceptable salt thereof, wherein:

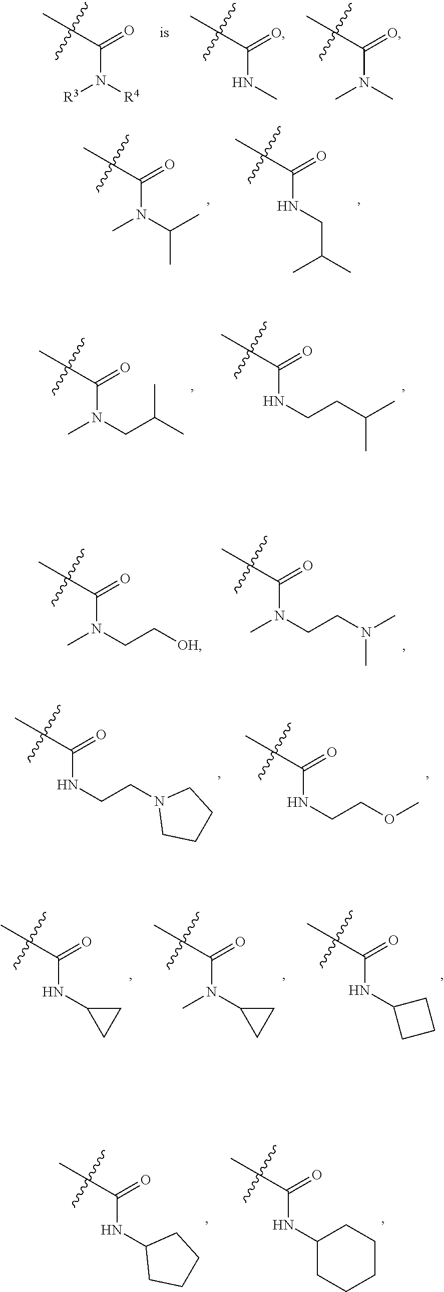

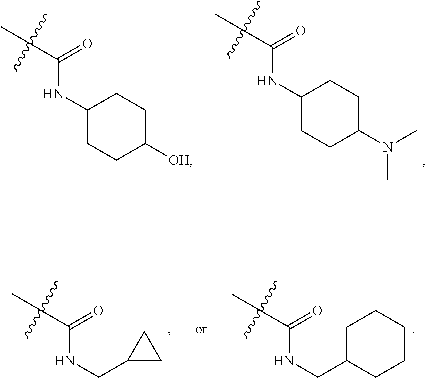 [SMILES: *C(=O)NC1CCC(O)CC1 ; *C(=O)NC1CCC(N(C)C)CC1 ; *C(=O)NCC1CC1 ; *C(=O)NCC1CCCCC1]

11. The compound of any one of claims 1-7, or a pharmaceutically acceptable salt thereof,
wherein:
R³ and R⁴ are taken together with the nitrogen atom to which they are attached to form a 5- to 6-membered heterocyclyl optionally substituted with 1-2 R⁷ groups, wherein the heterocyclyl optionally contains one additional heteroatom selected from N and O;
each R⁷ is independently halo, C₁-C₃ alkyl, —OH, C₁-C₃ alkoxy, or —NR^{6a}R^{6b}; and
each R^{6a} and R^{6b} is independently H or C₁-C₃ alkyl.

12. The compound of claim 11, or a pharmaceutically acceptable salt thereof, wherein:
R³ and R⁴ are taken together with the nitrogen atom to which they are attached to form a 5- to 6-membered heterocyclyl optionally substituted with one R⁷ group, wherein the heterocyclyl optionally contains one additional heteroatom selected from N and O; and
R⁷ is —OH, —CH₃, —OCH₃, or —N(CH₃)₂.

13. The compound of claim 12, or a pharmaceutically acceptable salt thereof, wherein:

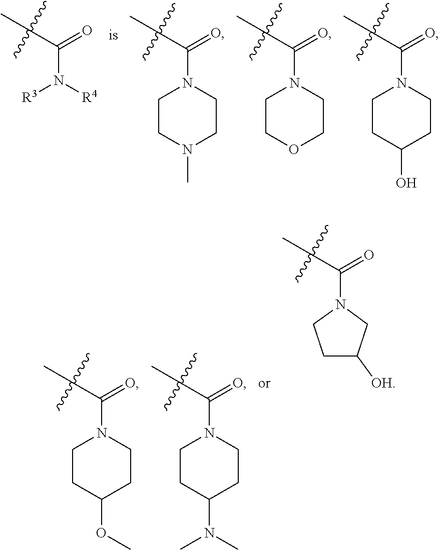

14. The compound of any one of claims 1-13, or a pharmaceutically acceptable salt thereof, wherein the compound is of Formula (II):

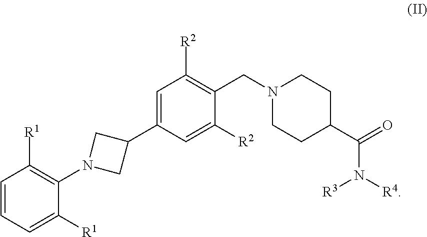

15. The compound of any one of claims 1-14, or a pharmaceutically acceptable salt thereof, wherein the compound is of Formula (IIIa): 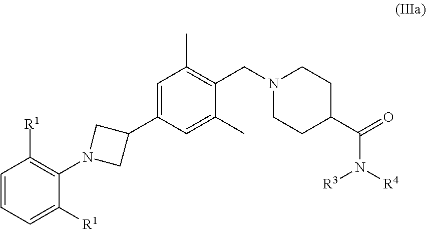
wherein:
R³ is H or C₁-C₆ alkyl;
R⁴ is H, C₁-C₆ alkyl, C₁-C₆ alkyl-OH, —(C₁-C₆ alkylene)-O—(C₁-C₆ alkyl), C₃-C₆ cycloalkyl, —(C₁-C₆ alkylene)(C₃-C₆ cycloalkyl), or —(C₁-C₆ alkylene)NR^{5a}R^{5b}, wherein each cycloalkyl is optionally substituted by 1-5 groups selected from halo, C₁-C₆ alkyl, C₁-C₆ haloalkyl, —OH, —NR^{6a}R^{6b}, and C₁-C₆ alkoxy;
R^{5a} and R^{5b} are independently H or C₁-C₆ alkyl;
or R^{5a} and R^{5b} are taken together with the nitrogen atom to which they are attached to form a 4-to 6-membered heterocyclyl; and
each R^{6a} and R^{6b} is independently H or C₁-C₆ alkyl.

16. The compound of any one of claims 1-14, or a pharmaceutically acceptable salt thereof, wherein the compound is of Formula (IVa): 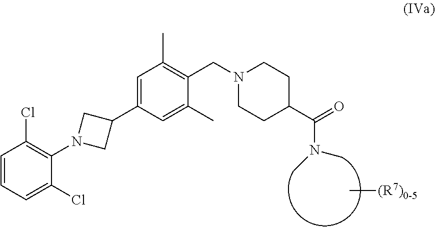
wherein 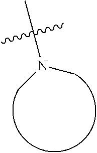
is a 4- to 6-membered heterocyclyl.

17. A compound selected from the compounds of Table 1 and pharmaceutically acceptable salts thereof.

18. A pharmaceutical composition comprising the compound of any one of claims 1-17, or a pharmaceutically acceptable salt thereof, and a pharmaceutically acceptable excipient.

19. A method of modulating sphingosine 1-phosphate receptor 5 (S1P5) comprising contacting S1P5 with an effective amount of the compound of any one of claims 1-17, or a pharmaceutically acceptable salt thereof, or the pharmaceutical composition of claim 18.

20. A method of treating a neurological disease in a subject in need thereof, comprising administering to the subject an effective amount of the compound of any one of claims 1-17, or a pharmaceutically acceptable salt thereof, or the pharmaceutical composition of claim 18, optionally wherein the neurological disease is Alzheimer's disease or multiple sclerosis.
